(19) United States
(12) Patent Application Publication
(10) Pub. No.: US 2024/0374528 A1
(43) Pub. Date: Nov. 14, 2024

(54) MULTIPARTICULATE DOSAGE FORMS COMPRISING DEUTETRABENAZINE

(71) Applicant: AUSPEX PHARMACEUTICALS, INC., Parsippany, NJ (US)

(72) Inventors: Mayank JOSHI, Parsippany, NJ (US); Sandeep PANDITA, Parsippany, NJ (US); Divyang PATEL, Parsippany, NJ (US); Soumen PATTANAYEK, Parsippany, NJ (US); Parag SHAH, Parsippany, NJ (US)

(21) Appl. No.: 18/692,074

(22) PCT Filed: Sep. 16, 2022
(86) PCT No.: PCT/US2022/076547

§ 371 Date: Mar. 14, 2024

Related U.S. Application Data
(60) Provisional application No. 63/245,299, filed on Sep. 17, 2021.

(51) Int. Cl. A61K 9/50; A61K 31/4745

(52) U.S. Cl. CPC A61K 9/5078; A61K 9/5084; A61K 31/4745

(57) ABSTRACT

Provided herein are modified release multiparticulate dosage forms containing deutetrabenazine for use in the treatment of, e.g., hyperkinetic movement disorders. The dosage forms are easily administered to a subject on a once-daily or twice daily basis, and provide a safe and effective pharmacokinetic profile.

### CROSS REFERENCE TO RELATED APPLICATIONS

This application claims the benefit of U.S. Provisional Application No. 63/256,299, filed Sep. 17, 2021, the entirety of which is incorporated by reference herein.

### TECHNICAL FIELD

The present disclosure pertains to multiparticulate dosage forms, manufacturing processes and methods of use of the multiparticulate dosage forms for treating hyperkinetic movement disorders deriving from conditions including Huntington's disease, tardive dyskinesia, levodopa-induced dyskinesia and dyskinesia in cerebral palsy.

### BACKGROUND

Deutetrabenazine ((RR,SS)-1,3,4,6,7,11b-hexahydro-9,10-di(methoxy-D3)-3-(2-methylpropyl)-2H-benzo[a]quinolizin-2-one) is a vesicular monoamine transporter type 2 (VMAT2). The biologically active metabolites formed from deutetrabenazine (alpha-dihydrodeutetrabenazine [α-deuHTBZ] and beta-dihydrodeutetrabenazine [β-deuHTBZ]), together identified as “deuHTBZ”, are potent inhibitors of VMAT2 binding. Deutetrabenazine exhibits an increased half-life of its active metabolites, relative to tetrabenazine (e.g., U.S. Pat. No. 8,524,733).

Deutetrabenazine (deu-TBZ) is approved by the U.S. Food and Drug Administration under the tradename AUSTEDO® for the treatment of chorea (involuntary muscle movements) associated with Huntington's disease (HD) and for the treatment of tardive dyskinesia (TD) in adults. AUSTEDO® dosage forms are orally administered twice-daily (bid), for total daily dosages of 12 mg or above of deutetrabenazine.

Several factors affect gastrointestinal absorption of orally administered drugs including solubility of the drug at various pH and the rate at which drug is released from the dosage form. Drug release rates for oral dosage forms are typically measured as rate of dissolution in vitro, i.e., a quantity of drug released from the dosage form per unit time in, for example, an FDA approved system. Such systems include, for example, United States Pharmacopeia (USP) dissolution apparati I, II and III.

The therapeutic window of a drug is the period when the plasma drug concentration is within the therapeutically effective plasma drug concentration range. Because the plasma drug concentration declines over time, however, multiple doses of drug dosage form must be administered at appropriate intervals to ensure that the plasma drug concentration remains within or, again rises to, the therapeutic window. At the same time, however, there is a need to avoid or minimize plasma drug concentrations that result in undesirable side effects.

Several dosage forms comprising deutetrabenazine are disclosed in U.S. Pat. No. 9,296,739. A dosage form that can deliver deutetrabenazine in a controlled manner over an extended period would enable a more advantageous dosing regimen, e.g., one that would permit once-daily (“qd”) administration and ease of oral administration, while maintaining the treatment effects currently realized by AUSTEDO®. A need exists for such alternative dosage forms.

### SUMMARY

Disclosed herein are sustained and controlled release multiparticulate dosage forms for once daily oral administration of deutetrabenazine to a subject in need thereof. Also disclosed are sustained and controlled release multiparticulate dosage forms for twice daily oral administration of deutetrabenazine to a subject in need thereof. The dosage forms, which may be packaged for example, in a capsule or pharmaceutical sachet package, are suitable for the target population.

### BRIEF DESCRIPTION OF THE DRAWINGS

FIG. 1A is an illustration of bead populations of the disclosure. It shows three options for the core of sustained release beads or the immediate release beads. The left figure represents granules, pellet or tablet comprising a first amount of micronized deutetrabenazine and first pharmaceutically acceptable excipient; the middle figure represents granules, pellet or tablet comprising a first amount of micronized deutetrabenazine and further optionally coated with an additional micronized deutetrabenazine dispersion; and the right figure represent a first amount of micronized deutetrabenazine dispersion coated inert particle.

FIG. 1B shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1C shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1D shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1E shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1F shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Ga shows possible sustained release beads, based on the core/immediate release beads in FIG. 1A. In the figure, a black layer represents a pH-independent polymer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Gb shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Ha shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Hb shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1I shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Ja shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Jb shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Ka shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Kb shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1L shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1M shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Na shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 1Nb shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 10a shows possible sustained release beads, based on the core/immediate release beads in FIG. 1A. In the figure, a black layer represents a pH-independent polymer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 10b shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

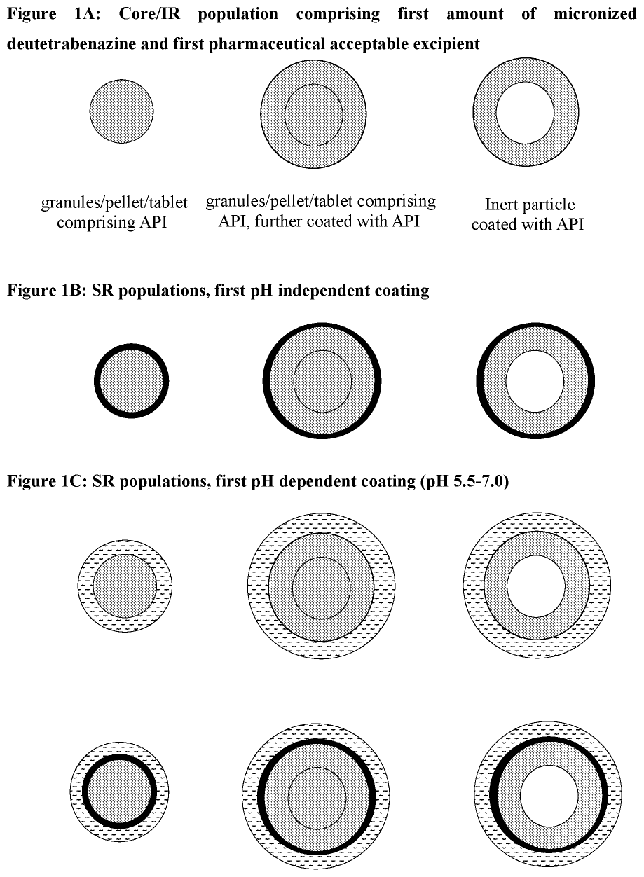

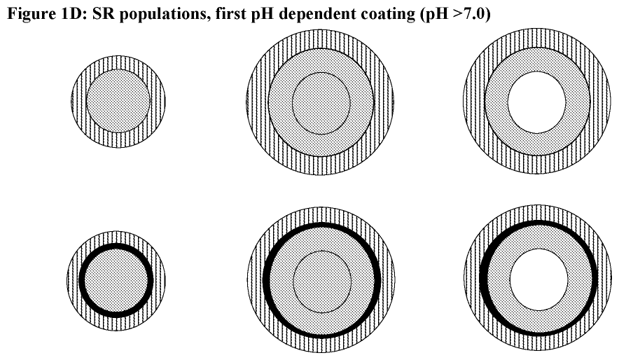

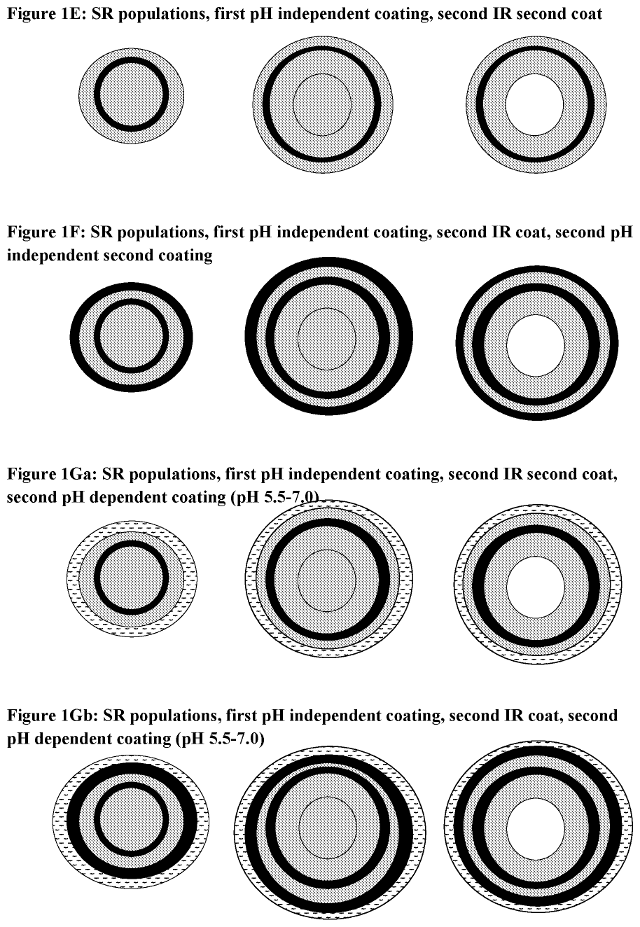

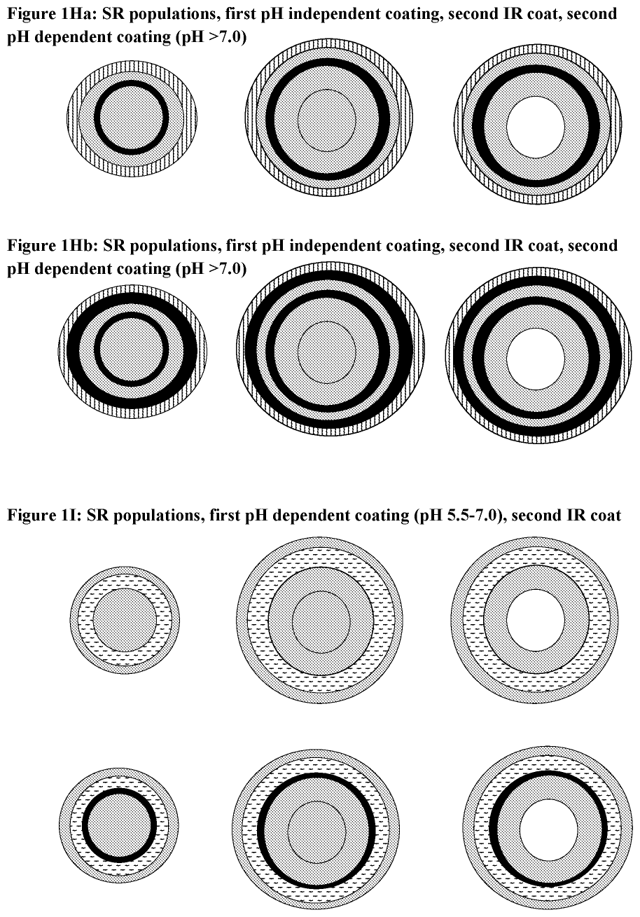

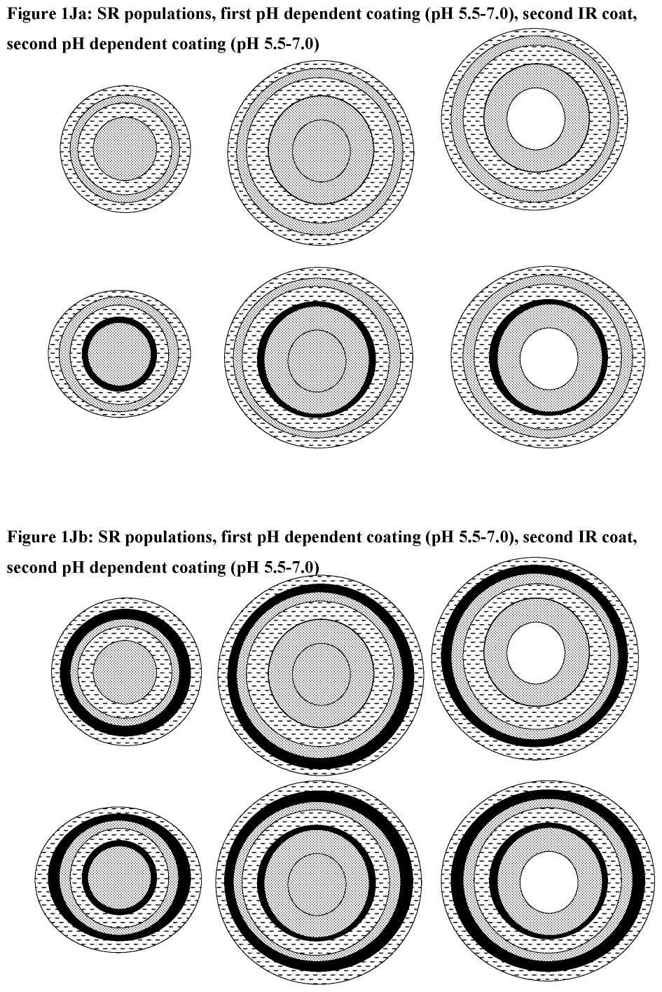

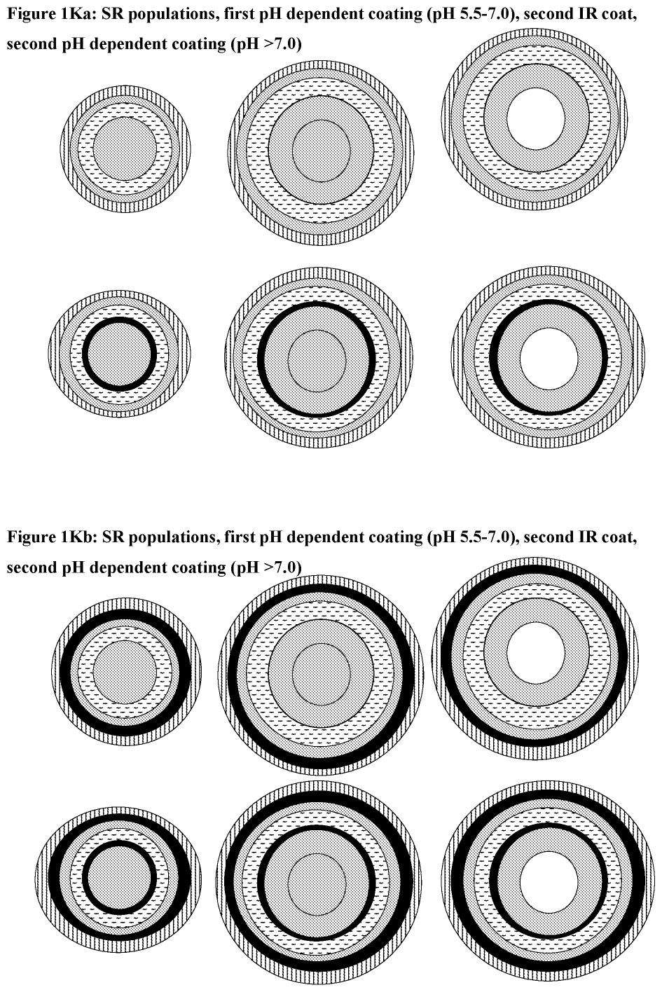

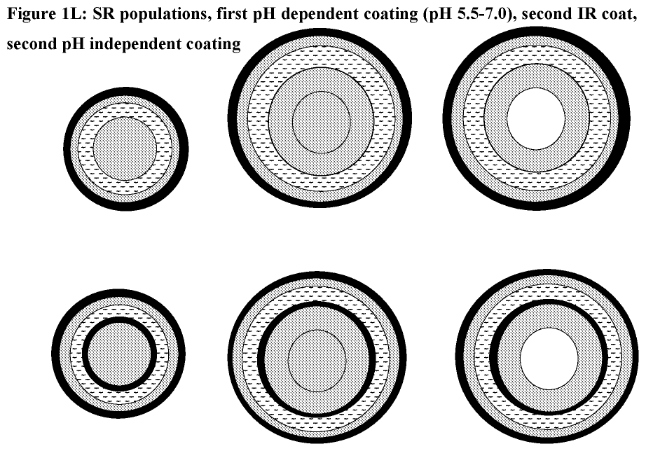

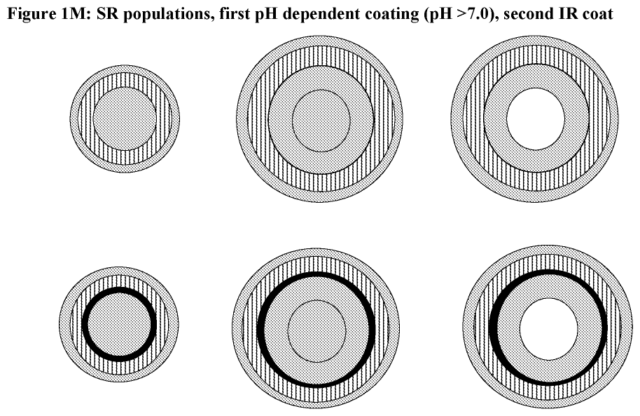

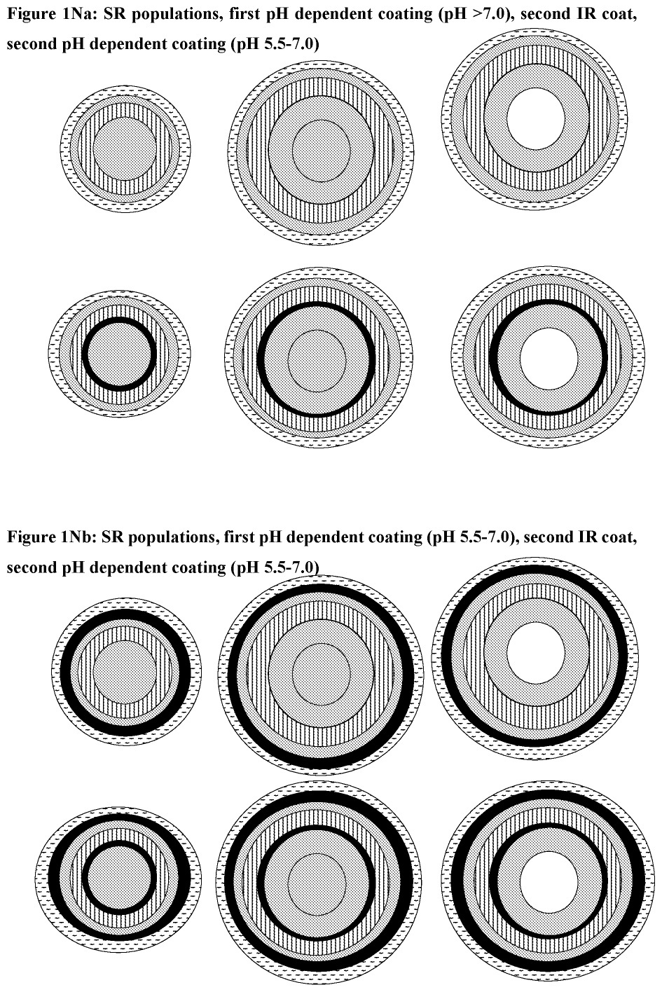

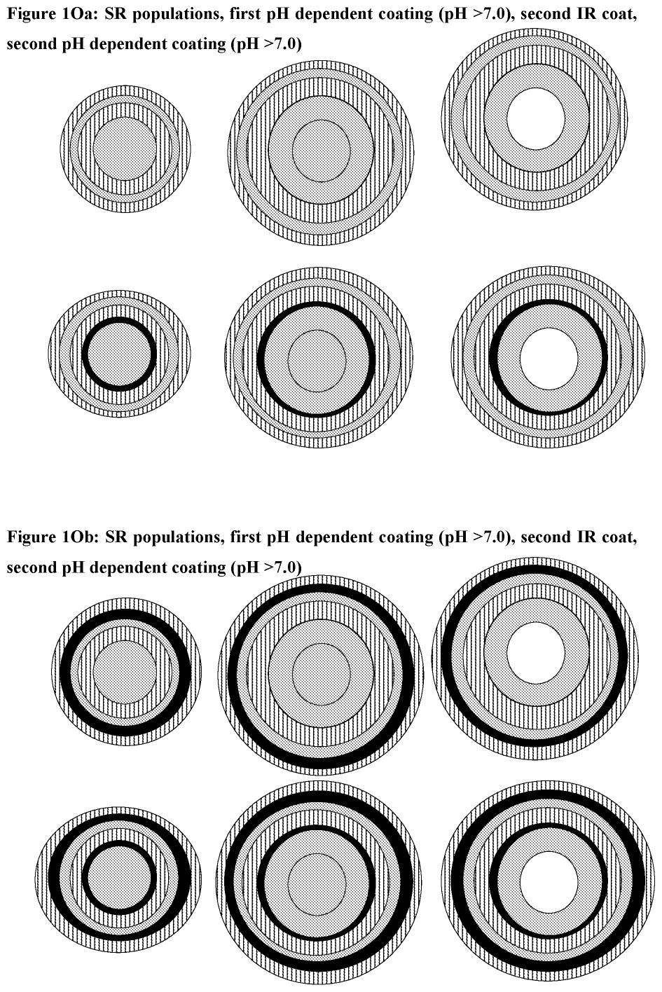

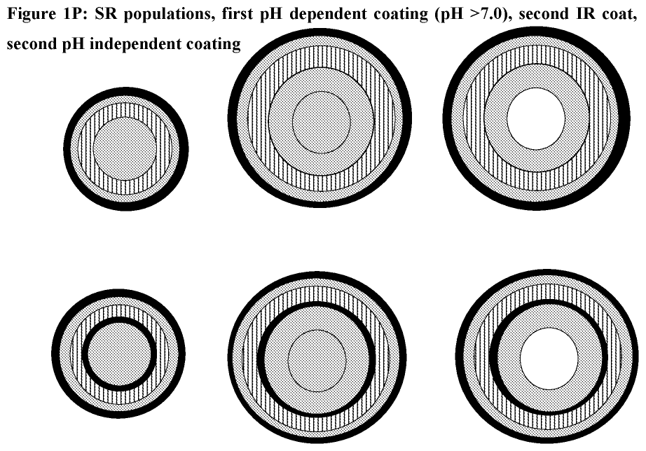

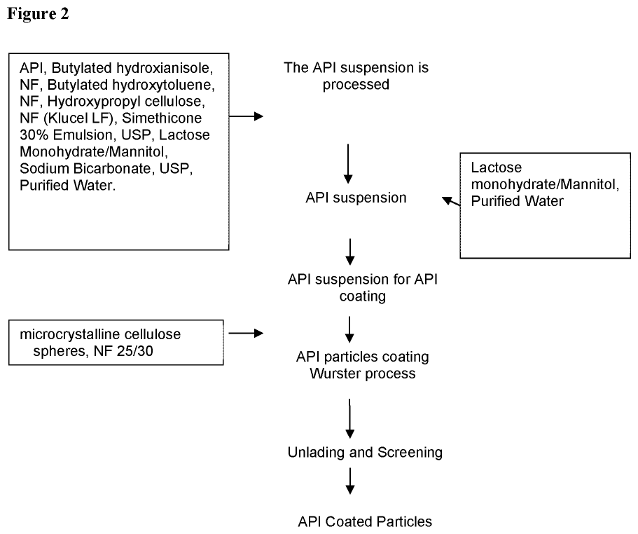

FIG. 1P shows possible sustained release beads, based on the core/immediate release beads in FIG. 1A. In the figure, a black layer represents a pH-independent polymer a dotted layer represents a pH-dependent polymer (sensitive to pH 5.5-pH 7) a striped layer represents a pH-dependent polymer (sensitive to pH>7). The oral dosage forms may comprise a single or a selection of any of the populations of sustained release beads demonstrated in the figure, optionally including a single or a selection of the immediate release populations demonstrated in FIG. 1A.

FIG. 2 provides a flowchart exemplifying the general manufacturing process for a micronized deuterated dispersion coated inert particle. The particle may serve as an immediate release bead or as a core for a sustained release bead.

### DETAILED DESCRIPTION OF ILLUSTRATIVE EMBODIMENTS

The present subject matter may be understood more readily by reference to the following detailed description, which forms a part of this disclosure. It is to be understood that this invention is not limited to the specific methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art.

As employed above and throughout the disclosure, the following terms and abbreviations, unless otherwise indicated, shall be understood to have the following meanings.

The singular forms “a,” “an,” and “the” may refer to plural articles unless specifically stated otherwise.

The term “about,” as used herein, is intended to qualify the numerical values which it modifies, denoting such a value as variable within a margin of +10%.

When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “compound”, “drug”, “pharmacologically active agent”, “active agent”, or “medicament” are used interchangeably herein to refer to a compound or compounds or composition of matter which, when administered to a subject (human or animal) induces a desired pharmacological and/or physiologic effect by local and/or systemic action. The active agent disclosed herein is preferably deutetrabenazine. “Deutetrabenazine” or “deu-TBZ” is a selectively deuterium-substituted, stable, non-radioactive isotopic form of tetrabenazine in which the six hydrogen atoms on the two O-linked methyl groups have been replaced with deuterium atoms (i.e. —OCD3 rather than —OCH3 moieties).

As used herein, “dosage form” refers to a drug form having multiparticulate properties wherein each bead population exhibits different properties.

The term “bead,” as used herein, refers to a discrete unit of the pharmaceutical formulation comprising at least a first amount of micronized deutetrabenazine and a first pharmaceutically acceptable excipient. In some embodiments, an “immediate release bead” refers to an immediate release formulation comprising a core, which can be formed from granules, a pellet or a tablet comprising the first amount of micronized deutetrabenazine and a first pharmaceutically acceptable excipient. In some embodiments, the immediate release bead comprises the core, e.g. granules, pellet, or tablet, further at least partially coated with a first amount of micronized deutetrabenazine and a first pharmaceutically acceptable excipient. In other embodiments, the immediate release bead comprises an inert particle, such as microcrystalline cellulose (MCC) or sugar particle, at least partially coated with a first amount of micronized deutetrabenazine and a first pharmaceutically acceptable excipient. In preferred embodiments, the sustained release beads disclosed herein comprise an immediate release core or immediate release particle (i.e. micronized deutetrabenazine containing granules, pellet, tablet, or coated inert particle) that is further coated with a first, and optionally a second, pH-independent polymer and/or pH-dependent polymer.

The terms “first amount of micronized deutetrabenazine and a first pharmaceutically acceptable excipient” and “first amount of micronized deutetrabenazine” as used herein refers to a dispersion of micronized deutetrabenazine in a pharmaceutically acceptable excipient, coating the core and/or an inert particle.

The terms “first coat” or “sustained release first coat” or “controlled release first coat” or “first pH-independent polymer coat” and “first pH-dependent polymer coat” as used herein, refers to a polymer coating layer selected from a pH-independent polymer coat, a pH-dependent polymer coat or a pH-independent polymer coat, further coated with a pH-dependent polymer coat, coating the first amount of micronized deutetrabenazine and the first pharmaceutically acceptable excipient.

The terms “second amount of micronized deutetrabenazine and a second pharmaceutically acceptable excipient” and “second amount of micronized deutetrabenazine” as used herein refers to a dispersion of micronized deutetrabenazine in a pharmaceutically acceptable excipient, coating the first coat.

The terms “second coat,” “sustained release second coat,” “controlled release second coat,” “second pH-independent polymer coat,” and “second pH-dependent polymer coat” as used herein interchangeably, refers to a polymer coating layer selected from a pH-independent polymer coat, a pH-dependent polymer coat or a pH-independent polymer coat further coated with a pH-dependent polymer coat, coating the second amount of micronized deutetrabenazine and a second pharmaceutically acceptable excipient.

The terms “first pharmaceutically acceptable excipient” and “first excipient” as used herein refer to a pharmaceutically acceptable excipient selected for use in the dispersion of the first amount of micronized deutetrabenazine, coating the core and/or an inert particle of the sustained release beads.

The terms “second pharmaceutically acceptable excipient” or “second excipient” as used herein interchangeably refers to a pharmaceutically acceptable excipient selected for use in the dispersion of the second amount of micronized deutetrabenazine, coating the first coat.

The terms “immediate release pharmaceutically acceptable excipient” and “immediate release excipient” as used herein refers to a pharmaceutically acceptable excipient selected for use in the dispersion of the first amount of micronized deutetrabenazine, coating the core and/or an inert particle of the immediate release beads.

The term “immediate release” (IR) as used herein refers to a pharmaceutical formulation, i.e. bead, which releases the active agent, i.e. deutetrabenazine, within about one hour post administration. Such release typically occurs in the upper gastrointestinal (GI) tract, for example in the stomach.

The term “sustained release” as used herein refers to a pharmaceutical formulation, i.e. bead, which releases the active agent, i.e. deutetrabenazine, over a prolonged period of time, typically from 1 to 12, or from 1 to 24 hr post administration. Such release typically occurs in the gastrointestinal (GI) tract, for example, in the upper intestine and/or lower intestines and/or colon.

“Controlled release” refers to a dosage form able to release active agent over an extended period for example, up to about 12 hours, 15 hours, 18 hours, 21 hours or up to about 24 hours. The active agent is preferably deutetrabenazine, as disclosed herein. Some of the active agent is released in the stomach (immediate release) and some in the small intestine and/or lower intestine/colon (sustained release). In some embodiments, the dosage form releases about 50 wt % of the active agent in the dosage form within 7 hours as measured in a USPIII apparatus, pH 7.2, In some embodiments, about 30% of deutetrabenazine is released within 2 hours, and about 65% is released within 6 hours and not less than (NLT) about 80% of deutetrabenazine is released within 10 hours, as measured in a USPIII dissolution device, pH 7.2. In some embodiments, about 25% of deutetrabenazine is released within 2 hours, and about 45% is released within 6 hours and NLT about 75% of deutetrabenazine is released within 10 hours as measured in a USPIII dissolution device, pH 7.2. In some embodiments, about 15% of deutetrabenazine is released within 2 hours, about 35% is released within 6 hours and NLT about 55% of deutetrabenazine is released within 10 hours as measured in a USPIII dissolution device, pH 7.2. In some embodiments, about 50% of micronized deutetrabenazine is released within 4 hours, as measured in a USPIII dissolution device, pH 7.2. In some embodiments not less than (NLT) about 80% of micronized deutetrabenazine is released within 8 hours, as measured in a USPIII dissolution device, pH 7.2.

The dosage forms as disclosed herein can be in the form of a capsule or otherwise packaged beads. A “capsule” is a dosage form encasing the bead populations, as disclosed herein. The capsule may be formed of gelatin (animal or vegetable) or other pharmaceutically acceptable material.

The gastrointestinal tract or “GI tract”, which extends from the oral cavity, through the esophagus, to the stomach and though the small intestine and colon to the anus, exhibits differing pH depending on the region and food status. The stomach is typically the first section of the GI tract in which disintegration and dissolution of drugs take place. The pH of the stomach is normally 1-3. The intestines are the main absorption site for nutrients and drugs. The small intestine has three distinct regions, duodenum, jejunum, and ileum. The entry of solid dosage forms into the small intestine is accompanied by a sharp pH increase because of the duodenal secretion of bicarbonate. Moreover, the literature suggests a subsequent increase of the pH value from about pH 5.5-6.8 in the duodenum to pH 6.8-8 in the terminal ileum. The pH values in the large intestine (including the colon), are slightly more acidic compared with the ileal pH values possibly due to the fermentation processes of the colonic microbiota (Koziolek, et al, J Pharma Sci; 104 (9) 2855-63).

As used herein, the terms “method of treatment” or “therapy” (as well as different forms thereof) include preventative (e.g., prophylactic), curative, or palliative treatment. As used herein, the term “treating” includes alleviating or reducing at least one adverse or negative effect or symptom of a condition, disease or disorder. This condition, disease or disorder may refer to hyperkinetic movement disorder, such as, but not limited to, chorea associated with Huntington's disease, tardive dyskinesia, Tourette syndrome, dystonia, dyskinesia in cerebral palsy (DCP) and levodopa-induced dyskinesia (LID) in Parkinson's disease.

The term “administering” means providing to a patient the pharmaceutical composition or dosage form (used interchangeably herein) of the present invention.

The terms “subject”, “individual”, and “patient” are used interchangeably herein, and refer to a human, to whom treatment, including prophylactic treatment, with the dosage form according to the present invention, is provided.

“Pharmaceutically acceptable” refers to those compounds, materials, compositions, and/or excipients which are, within the scope of sound medical judgment, suitable for contact with the tissues of human beings without excessive toxicity, irritation, allergic response, or other problem complications commensurate with a reasonable benefit/risk ratio.

“Microparticles” refers to particles, for example deutetrabenazine particles, with a particle size (i.e. diameter) below 1 mm. In one embodiments, the median diameter (D₅₀) of the microparticles is from about 0.05 to about 100 μm. In another embodiment, D₅₀ of the microparticles is from about 0.05 to about 50 μm. In another embodiment, the D₅₀ of the microparticles is from about 1 μm to about 30 μm, or about 1 μm to about 25 μm, or about 5 μm to about 30 μm, or about 1 μm to about 20 μm, or about 5 μm to about 25 μm, or about 10 μm to about 20 μm. In one embodiment, the deutetrabenazine microparticles have a particle size distribution of about 1 μm to about 30 μm in diameter. In another embodiment, the deutetrabenazine microparticles have a D₉₀ of 15 μm (i.e., 90% of the particles have a diameter less than or equal to 15 μm). In another embodiment, the deutetrabenazine microparticles have a D₅₀ 10 μm (i.e., 50% of the particles have a diameter greater than 10 μm and 50% of the particles have a diameter less than or equal to 10 μm). In yet another embodiment, the deutetrabenazine microparticles have a D₁₀ of 3 μm (i.e., 10% of the particles have a diameter less than 3 μm).

The terms D₉₀, D₅₀ or D₁₀ are well understood in the art. The particle size distribution of the microparticles (i.e. the diameters) can be determined by one with skill in art using conventional methods, for example, dynamic or static light-scattering of an aqueous dispersion of the microparticle composition. The D₉₀ and D₁₀ values, like the D₅₀ value, can be calculated from the particle size distribution of the microparticles. For example, a D₉₀ of 15 μm, means that 90% (by volume) of the particles have a size less than or equal to 15 μm. A D₅₀ of 10 μm, means that 50% (by volume) of the particles have a size less than or equal to 10 μm. A D₁₀ of 3 μm, means that 10% (by volume) of the particles have a size less than or equal to 3 μm. The terms may be combined to define a particle size distribution (PSD).

Particle size distribution is determined by means of laser diffractometry. More specifically, the particle size distribution was determined using a Mastersizer 3000 from Malvern Instruments. The particle size determination may be carried out as a wet or dry measurement depending on the sample.

Although constant-release dosage forms have been proven effective for many different drug therapies, there are clinical situations where these have not been entirely satisfactory. It has been observed that for some patients, the therapeutic effectiveness of the drug decreases below the therapeutically effective threshold before the end of the desired therapy period despite the maintenance of substantially constant drug release that would be expected to provide continued effectiveness.

Dosage forms of the disclosure provide for improved release control, as compared to dosage forms previously described. For example, it has been discovered that a better release control profile is obtained when a mixture of cellulose acetates is used in the dosage form, as compared to dosage forms not including a mixture of cellulose acetates, for example, as compared to dosage forms including ethylcellulose. Dosage forms of the disclosure also perform better in alcohol “dose dumping” experiments. “Dose dumping” occurs when a relatively large amount of drug in a controlled or sustained release formulation is quickly released, resulting in a potentially toxic amount of drug entering systemic circulation. Dosage forms of the disclosure result in less dose dumping as compared to dosage forms previously described, thereby lowering the risk of associated adverse events. In some embodiments, provided are dosage forms that are resistant to alcohol induced dose dumping.

Provided herein are controlled release oral dosage forms for once daily administration of deutetrabenazine comprising a population of sustained release beads; wherein the sustained release beads comprise a core comprising a first amount of micronized deutetrabenazine and a first pharmaceutically acceptable excipient, and further comprising a first coat selected from pH-independent polymer coat, a pH-dependent polymer coat or a pH-independent polymer coat further coated with a pH-dependent polymer coat. Optionally, the sustained release beads further comprise a film coat, comprising a mixture of hydrophilic and hydrophobic polymers.

Also provided herein are controlled release oral dosage forms for twice daily administration of deutetrabenazine comprising a population of sustained release beads; wherein the sustained release beads comprise a core comprising a first amount of micronized deutetrabenazine and a first pharmaceutically acceptable excipient, and further comprising a first coat selected from pH-independent polymer coat, a pH-dependent polymer coat or a pH-independent polymer coat further coated with a pH-dependent polymer coat. Optionally, the sustained release beads further comprise a film coat, comprising a mixture of hydrophilic and hydrophobic polymers.

The core of the sustained release beads may be one of several forms, for example, a) immediate release granules, immediate release pellets or immediate release tablets comprising a first amount of micronized deutetrabenazine and a first pharmaceutically acceptable excipient or b) an inert particle coated with a first amount of the micronized deutetrabenazine and a first pharmaceutically acceptable excipient. In some embodiments, the core of the sustained release beads comprises immediate release granules, immediate release pellets or immediate release tablets. In some embodiments, the core of the sustained release beads comprises a coated inert particle.

In some embodiments, the sustained release beads further comprise a second amount of deutetrabenazine and a second pharmaceutically acceptable excipient coat, on top of the first coat. In some embodiments, the sustained release beads coated with the second amount of deutetrabenazine and a second pharmaceutically acceptable excipient, further comprise a second coat selected from a pH-independent polymer coat, a pH-dependent polymer coat, or a pH-independent polymer coat further coated with a pH-dependent polymer coat.

In some embodiments, the dosage forms of the disclosure include one or more populations of the sustained release beads.

In other embodiments, the dosage form includes a population of the sustained release beads and a population of immediate release beads; wherein the population of immediate release beads comprises a) immediate release granules, immediate release pellets or immediate release tablets comprising a first amount of micronized deutetrabenazine and a first pharmaceutically acceptable excipient or b) an inert particle coated with a first amount of micronized deutetrabenazine and a first pharmaceutically acceptable excipient. In some embodiments of the dosage form, the core per se of the sustained release particles serves as the population of immediate release beads. Therefore, in some embodiments, the first amount of micronized deutetrabenazine and/or the first pharmaceutically acceptable excipient, independently or in combination with the second amount of micronized deutetrabenazine and/or the second pharmaceutically acceptable excipient, are the same in the core of the sustained release beads or cumulatively in the sustained release beads and in the immediate release beads. However, the first amount of micronized deutetrabenazine and/or the first pharmaceutically acceptable excipient, independently or in combination with the second amount of micronized deutetrabenazine and/or the second pharmaceutically acceptable excipient, may be different in the core of the sustained release beads or cumulatively in the sustained release beads and in the immediate release beads.

For clarity, the amount of the first and optionally second micronized deutetrabenazine, as well as well as the amount and selection of the first and optionally second excipients, are independently selected for each of the dispersions in the sustained release particles and immediate release particles. In preferred embodiments, the deutetrabenazine is provided as deutetrabenazine microparticles. In various embodiments, the micronized deutetrabenazine in the first amount of micronized deutetrabenazine or the second amount of micronized deutetrabenazine or the amount of micronized deutetrabenazine in the immediate release beads, is present in at a concentration of 5 wt %-80 wt %, or 10 wt %-80 wt %, or 10 wt %-70 wt %, 20 wt %-60 wt %, 5 wt %-30 wt %, or 50 wt %-80 wt % of weight of the core of the sustained release beads or of the immediate release beads, respectively.

The micronized deutetrabenazine is present in the first amount of micronized deutetrabenazine, and in the optional second amount of micronized deutetrabenazine and/or in the optional immediate release beads, together with a first and optionally a second and/or immediate release pharmaceutically acceptable excipient. In these embodiments, the core (i.e. first and or second) pharmaceutically acceptable excipient and the immediate release pharmaceutically acceptable excipient each independently comprises at least one of an antioxidant, a binder, a filler, a surfactant, an anti-foaming agent, or any combinations thereof. In some embodiments, the first, second and/or the immediate release pharmaceutically acceptable excipient, each independently, comprises an antioxidant, a binder, a filler, a surfactant, and/or an anti-foaming agent.

In some embodiments, the first, second and/or the immediate release pharmaceutically acceptable excipient, independently, comprises an antioxidant, which may be a water-insoluble antioxidant. The water-insoluble antioxidant comprises butylated hydroxytoluene (BHT), butylated hydroxyanisole (BHA), propyl gallate, 6-ethoxy-1,2-digydro-2,2,4-trimethylquinoline (ethoxyquin), nordihydroguaiaretic acid (NDGA), sodium metabisulfite (SMB), a tocopherol or combinations thereof. In some embodiments, the water-insoluble antioxidant comprises butylated hydroxytoluene (BHT), butylated hydroxyanisole (BHA) or a combination thereof. The water-insoluble antioxidant may be present in the first amount of micronized deutetrabenazine and the first pharmaceutically acceptable excipient or the second amount of micronized deutetrabenazine and the second pharmaceutically acceptable excipient or in the immediate release bead, independently, at a concentration of 0.1 wt %-1.0 wt % of the weight of the core or of the sustained release bead, or the immediate release bead, respectively.

In some embodiments, the first, second and/or the immediate release pharmaceutically acceptable excipient, independently, comprises a binder. The binder may be selected from the group consisting of a water-soluble binder, a water-insoluble binder and combinations thereof. In some embodiments, the binder comprises a water-soluble binder, which includes hydroxypropyl cellulose, hydroxypropyl methylcellulose, polyvinyl pyrrolidone, polyvinyl alcohol, a polyacrylic acid polymer, polyether, carbohydrate polymer (natural or synthetic) or combinations thereof. In some embodiments, the binder comprises a water-insoluble polymer, which includes crospovidone, copovidone, microcrystalline cellulose, croscarmellose sodium, starch, sodium starch glycolate, colloidal silica, silica, ethyl cellulose, lactic acid polymer, a lactic acid and glutamic acid copolymer, polyvinyl acetate or combinations thereof. In some embodiments, the binder comprises a polyether, including polyethylene glycol (PEG). The binder may be present in the first amount of micronized deutetrabenazine and the first pharmaceutically acceptable excipient, or the second amount of micronized deutetrabenazine and the second pharmaceutically acceptable excipient, or in the immediate release bead, independently, at a concentration of 0.5 wt %-10.0 wt % of the weight of the core, or of the sustained release bead, or the immediate release bead, respectively.

In some embodiments, the first, second and/or the immediate release pharmaceutically acceptable excipient, independently, comprises a filler. The filler may be a saccharide, a disaccharide, a polysaccharide, a polyalcohol, microcrystalline cellulose, natural and synthetic gums, pregelatinized starch, polyvinylpyrrolidone, cellulose derivatives, dibasic calcium phosphate, kaolin, inorganic salts, calcium carbonate, sodium bicarbonate, sodium carbonate, and combinations thereof. In some embodiments, the filler comprises microcrystalline cellulose, a saccharide, a polyalcohol or a combination thereof. In some embodiments, the saccharide is lactose. In some embodiments, the polyalcohol is mannitol. The filler may be present in the first amount of micronized deutetrabenazine and the first pharmaceutically acceptable excipient or the second amount of micronized deutetrabenazine and the second pharmaceutically acceptable excipient or in the immediate release bead, independently, at a concentration of 5.0 wt %-50.0 wt % of the weight of the core or of the sustained release bead, or the immediate release bead, respectively.

In some embodiments, the first, second and/or the immediate release pharmaceutically acceptable excipient, independently, comprises a surfactant. The surfactant may include sodium lauryl sulfate, sodium dodecyl sulfate, sodium laureth sulfate, docusate sodium, polysorbate, tween, polyoxyethylene 15 hydroxy stearate, polyoxyethylene castor oil derivatives, polyoxyethylene stearates, sorbitan fatty acid esters, polyoxyethylene alkyl ethers, polyoxyethylene nonylphenol ether or combinations thereof. In some embodiments, the surfactant includes sodium lauryl sulfate. The surfactant may be present in the first amount of micronized deutetrabenazine and the first pharmaceutically acceptable excipient or the second amount of micronized deutetrabenazine and the second pharmaceutically acceptable excipient or in the immediate release bead, independently, at a concentration of 2.0 wt %-12.0 wt % of the weight of the core or of the sustained release bead, or the immediate release bead, respectively.

In some embodiments, the first, second and/or the immediate release pharmaceutically acceptable excipient, independently, comprises an anti-foaming agent. The anti-foaming agent may include insoluble oils, polydimethylsiloxanes and other silicones, certain alcohols, stearates, glycols and combinations thereof, preferably simethicone, dimethicone, tilactase or peppermint oil. The anti-foaming agent may be present in the first amount of micronized deutetrabenazine and the first pharmaceutically acceptable excipient or the second amount of micronized deutetrabenazine and the second pharmaceutically acceptable excipient or in the immediate release bead, independently, at a concentration of 0.3 wt %-3.0 wt % of the weight of the core or of the sustained release bead, or the immediate release bead, respectively.

The core of the sustained release beads comprising the first amount of the deutetrabenazine and first amount of the excipient may be coated with a first coat selected from a pH-independent polymer coat and/or a pH-dependent polymer. In some embodiments, the sustained release beads may further comprise a second amount of deutetrabenazine and a second pharmaceutically acceptable excipient and further a second coat selected from a pH-independent polymer and/or a pH-dependent polymer. In some embodiments, the first and optionally the second coat of the sustained release beads, independently, include a pH-independent polymer coat. The pH-independent polymer coat may be a cellulose acetate, a mixture of cellulose acetates, ethylcellulose or a mixture of ethylcellulose and polyethylene glycol. In some embodiments, the pH-independent polymer coat comprises ethylcellulose. In some embodiments, the pH-independent polymer coat comprises cellulose acetate. In some embodiments, the pH-independent polymer coat comprises a mixture of cellulose acetate NF 398-10 and cellulose acetate 320S. In some embodiments, the pH-independent polymer coat comprises a mixture of cellulose acetate and polyethylene glycol. The first and optionally the second coat of the sustained release beads, independently, may further include a pH-dependent polymer coat coating the pH-independent polymer coat.

In some embodiments, the first and optionally the second coat of the sustained release beads, independently, include a pH-dependent polymer coat coating the core or the second amount of micronized deutetrabenazine and a second pharmaceutically acceptable excipient, respectively. In some embodiments, the pH-dependent polymer coat is formulated to dissolve at a pH of about 5.0-7.0, for example in the upper small intestine of a human subject. The pH-dependent polymer coat may be methacrylic acid-ethyl acrylate copolymer, hydroxypropylmethyl cellulose phthalate (HPMCP), alginates, carboxymethylcellulose, or a combination thereof. In some embodiments, the pH-dependent polymer coat comprises methacrylic acid-ethyl acrylate copolymer.

In some embodiments, the pH-dependent polymer coat is formulated to dissolve at a pH above 7.0, for example in the large intestine or colon of a human subject. In that case, the pH-dependent polymer coat may include cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac, or combinations thereof. In some embodiments, the pH-dependent polymer coat comprises methacrylic acid-methyl methacrylate copolymer.

For clarity, the amount and/or the selection of the pH-independent or pH-dependent polymer coat are independent for each the first and the second coat.

The pH-independent polymer in the first and optionally the second coat, independently, or the pH-dependent polymer in the first and optionally the second coat, independently, may further include a pharmaceutically acceptable plasticizer. The plasticizer may include triethyl citrate (TEC), triacetin, acetyl tributyl citrate, acetyl triethyl citrate, glycerin, a polyethylene glycol, polyethylene glycol monomethyl ether, propylene glycol, sorbitol sorbitan solution, castor oil, diacetylated monoglycerides, dibutyl sebacates, diethyl phthalate or combinations thereof. In some embodiments, the plasticizer comprises triethyl citrate. In some embodiments, the pH-independent polymer coat or the pH-dependent polymer coat is present on the sustained release bead at a concentration of 15.0 wt %-50.0 wt % of the weight of the sustained release bead. The pH-independent polymer coat or the pH-dependent polymer coat may be present on the sustained release bead at a concentration of 20.0 wt %-40.0 wt % of the weight of the sustained release bead. For clarity, the amount and/or the selection of the plasticizer, are independent for each the first and optionally the second coat the sustained release particles.

In some embodiments, the dosage form disclosed herein, comprises a total of 6 mg-72 mg of micronized deutetrabenazine. In some embodiments, the dosage form comprises a total of 6 mg, or 12 mg, or 18 mg, or 24 mg, or 30 mg, or 36 mg, or 42 mg or 48 mg of micronized deutetrabenazine.

The dosage form disclosed herein may consist essentially of a population of sustained release beads comprising a pH-independent polymer coat or a population of sustained release beads comprising a pH-independent polymer coat further coated with a pH-dependent polymer coat. The dosage form may be a capsule, a sachet or the like.

In some embodiments, the dosage form consists essentially of a population of sustained release beads comprising:
a) a core comprising first amount of micronized deutetrabenazine and a first pharmaceutically acceptable excipient; wherein the first pharmaceutically acceptable excipient comprises: an antioxidant, a water-soluble binder, an anti-foaming agent, a filler, and a surfactant;
b) a first pH-independent polymer coat coating the core; and optionally further comprising
c) a capsule shell or pharmaceutical sachet packaging.

In some embodiments, the core comprises an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate, or mixtures thereof, and a surfactant comprising sodium lauryl sulfate.

The core may be in the form of immediate release granules, immediate release pellet or immediate release tablet or tablets or an inert particle coated with the first amount of the micronized deutetrabenazine and the first pharmaceutically acceptable excipient. In some embodiments, the first pH-independent polymer coat comprises ethylcellulose. In some embodiments, the pH-independent polymer coat comprises ethylcellulose, polyethylene glycol and triethyl citrate, and optionally further comprises povidone. In some embodiments, the pH-independent polymer coat comprises a mixture of cellulose acetate NF 398-10 and cellulose acetate 320S. In some embodiments, the pH-independent polymer coat comprises cellulose acetate and optionally polyethylene glycol.

In various embodiments, the dosage form comprises a population of sustained release beads and further comprises a population of immediate release beads. The immediate release beads comprise one of a) immediate release granules, immediate release pellet or immediate release tablet comprising a first amount of micronized deutetrabenazine and a first pharmaceutically acceptable excipient or b) an inert particle coated with a first amount of micronized deutetrabenazine and a first pharmaceutically acceptable excipient. In some embodiments, the immediate release beads include (b).

In some embodiments, the dosage form comprises a population of immediate release beads and a population of sustained release beads, the sustained release beads comprising
a) a core comprising a first amount of micronized deutetrabenazine and the first pharmaceutically acceptable excipient; wherein the first pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate or mixtures thereof, and a surfactant comprising sodium lauryl sulfate;
b) a first pH-dependent polymer coat sensitive to pH 5.5-pH 7 coating the core.

The first pH-dependent polymer coat may include methacrylic acid-ethyl acrylate copolymer, hydroxypropylmethyl cellulose phthalate (HPMCP), alginates, carboxymethylcellulose, or a combination thereof. Without wishing to be bound to any particular theory, the pH-dependent polymer coat comprising methacrylic acid and ethyl acrylate copolymer, and triethyl citrate is sensitive in a pH of about 5.5 to about 7, thereby targeting the small intestine.

In some embodiments, the dosage form comprises a population of sustained release beads comprising
a) a core comprising a first amount of micronized deutetrabenazine and a first pharmaceutically acceptable excipient; wherein the first pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate or mixtures thereof, and a surfactant comprising sodium lauryl sulfate;
b) a first pH-dependent polymer coat that is sensitive to about pH 7 to about pH 8 coating the core.

The first pH-dependent polymer coat sensitive to pH>7.0 may be cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac, or combinations thereof. Without wishing to be bound to any particular theory, the first pH-dependent polymer coat comprises methacrylic acid and methyl acrylate copolymer and triethyl citrate and is sensitive to a pH of about 7 to about 8, thereby dissolving in the large intestine/colon.

The core of the aforementioned sustained release beads comprises a) immediate release granules, an immediate release pellet or an immediate release tablet comprising the first amount of micronized deutetrabenazine and the first pharmaceutically acceptable excipient or b) an inert particle coated with a first amount of micronized deutetrabenazine and the first pharmaceutically acceptable excipient. In some embodiments, the core comprises (b).

In some embodiments, the dosage forms disclosed herein include a population of immediate release beads and a population of sustained release beads, the sustained release beads having a pH-dependent coating that dissolves at pH 5.5-7.

In some embodiments, the dosage forms disclosed herein include a population of immediate release beads and a population of sustained release beads, the sustained release beads having a pH-dependent coating that dissolves at pH>7.

In some embodiments, the dosage forms disclosed herein include a population of immediate release beads and two populations of sustained release beads, one population of the sustained release beads having a pH-dependent coating that dissolves at pH 5.5-7.0, and a second population of sustained release beads having a pH-dependent coating that dissolves at pH>7.

The dosage forms disclosed herein may be in the form of a capsule, comprising a capsule shell and at least one population of sustained release beads, optionally further comprising a population of immediate release beads. Alternatively, the dosage forms disclosed herein may be in the form of a sachet, comprising a sachet package and at least one population of sustained release beads, optionally further comprising a population of immediate release beads.

In some embodiments, about 50% of micronized deutetrabenazine is released within 7 hours, as measured in a USPIII dissolution device, pH 7.2. In some embodiments, about 30% of micronized deutetrabenazine is released within 2 hours, and about 65% is released within 6 hours and not less than (NLT) about 80% of micronized deutetrabenazine is released within 10 hours, as measured in a USPIII dissolution device, pH 7.2. In some embodiments, about 25% of micronized deutetrabenazine is released within 2 hours, and about 45% is released within 6 hours and NLT about 75% of micronized deutetrabenazine is released within 10 hours as measured in a USPIII dissolution device, pH 7.2. In some embodiments, about 15% of micronized deutetrabenazine is released within 2 hours, about 35% is released within 6 hours and NLT about 55% of micronized deutetrabenazine is released within 10 hours as measured in a USPIII dissolution device, pH 7.2.

In some embodiments, about 50% of micronized deutetrabenazine is released within 4 hours, as measured in a USPIII dissolution device, pH 7.2. In some embodiments not less than (NLT) about 80% of micronized deutetrabenazine is released within 8 hours, as measured in a USPIII dissolution device, pH 7.2.

Further provided herein, are methods useful in treating VMAT2 mediated disorders. In some embodiments, the method of treating a VMAT2 mediated disorder comprises orally administering to a patient in a need thereof, a controlled release dosage form as disclosed herein. Also provided herein, are methods useful in treating VMAT2 mediated disorders. In some embodiments, the method of treating a VMAT2 mediated disorder comprises orally administering to a patient in a need thereof, a sustained release dosage form as disclosed herein. The VMAT2 mediated disorder may be a hyperkinetic movement disorder. The hyperkinetic movement disorder may be a chronic disorder, for example Huntington's disease, tardive dyskinesia, and dyskinesia in cerebral palsy.

Further provided herein is a process for manufacturing the immediate release beads or the core of the sustained release beads, comprising the steps of
a) providing a dispersion of a first amount of micronized deutetrabenazine with a first pharmaceutically acceptable excipient, wherein the first pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate, or mixture thereof;
b) forming immediate release granules, an immediate release pellet or an immediate release tablet from the dispersion of a); or coating an inert particle with the dispersion of a);
thereby generating the immediate release beads or the core of the sustained release beads.

Further provided is a process for manufacturing the sustained release beads comprising the steps of
a) providing a micronized deutetrabenazine dispersion comprising a first amount of micronized deutetrabenazine and a first pharmaceutically acceptable excipient, wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate or mixtures thereof;
b) providing a core, wherein the core comprises immediate release granules, immediate release pellet or immediate release tablet comprising the dispersion of a); or an inert particle coated with the dispersion of a);
c) coating the core of b) with a first pH-independent polymer coating, a first pH-dependent polymer coating or with a first pH-independent polymer coating and a pH-dependent polymer coating;
thereby generating sustained release beads.

In some embodiments, the process further comprises:
d) coating the sustained release beads with a second micronized deutetrabenazine dispersion comprising a second amount of micronized deutetrabenazine and a second pharmaceutically acceptable excipient, wherein the second pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate or mixtures thereof;
thereby generating sustained release beads comprising a second amount of immediate release micronized deutetrabenazine and a second pharmaceutically acceptable excipient.

In some embodiments, the process further comprises:
e) coating the sustained release beads comprising the second immediate release micronized deutetrabenazine and a second pharmaceutically acceptable excipient with a second coat selected from a pH-independent polymer, a pH-dependent polymer and a pH-independent polymer and a pH-dependent polymer coat;
thereby generating sustained release beads comprising a second sustained release coat.

In some embodiments, the process further comprises, subsequent to any one of steps c-e, coating with a film coat, the film coat comprising a mixture of hydrophilic and hydrophobic polymers.

It has been surprisingly discovered that oral dosage forms comprising deutetrabenazine that exhibit a desirable rate of release and hence a desirable pharmacokinetic (PK) profile for an extended time can be achieved. In some embodiments, the presently disclosed multiparticulate dosage forms when administered orally to a subject on a once daily basis (qd) provide a pharmacokinetic profile that is comparable, e.g., bioequivalent, to that of the AUSTEDO® dosage forms administered twice daily (bid). In other embodiments, the presently disclosed multiparticulate dosage forms when administered orally to a subject on a twice daily basis (bid) provide a pharmacokinetic profile that is comparable, e.g., bioequivalent, to that of the AUSTEDO® dosage forms administered twice daily (bid).

Provided herein is a sustained release oral dosage form for once daily administration of deutetrabenazine comprising a population of sustained release beads; wherein the sustained release beads comprise a core comprising a first amount of micronized deutetrabenazine and a first pharmaceutically acceptable excipient; and further comprising a first coat selected from pH-independent polymer coat, a pH-dependent polymer coat or a pH-independent polymer coat further coated with a pH-dependent polymer coat. Also provided herein is a sustained release oral dosage form for twice daily administration of deutetrabenazine comprising a population of sustained release beads; wherein the sustained release beads comprise a core comprising a first amount of micronized deutetrabenazine and a first pharmaceutically acceptable excipient; and further comprising a first coat selected from pH-independent polymer coat, a pH-dependent polymer coat or a pH-independent polymer coat further coated with a pH-dependent polymer coat. In some embodiments, the core comprises immediate release granules, immediate release pellet or immediate release tablet that comprises the first amount micronized deutetrabenazine and a first pharmaceutically acceptable excipient. The micronized deutetrabenazine and pharmaceutically acceptable excipient may be a deutetrabenazine dispersion. In some embodiments, the core comprises an inert particle for example, a microcrystalline cellulose particle. Such particles are well known to the formulator skilled in the art. In such embodiments, the core comprises an inert particle coated with the first amount of micronized deutetrabenazine and a first pharmaceutically acceptable excipient dispersion.

In some embodiments, the dosage form further comprises a population of immediate release beads; wherein the population of immediate release beads comprises a) immediate release granules, immediate release pellet or immediate release tablet comprising a first amount of micronized deutetrabenazine and a first pharmaceutically acceptable excipient or b) an inert particle coated with a first amount of micronized deutetrabenazine and a first pharmaceutically acceptable excipient. In some embodiments, a portion of the immediate release granules, pellet or tablet or inert particle of the immediate release beads serves as the core of the sustained release beads.

It is now related that the dosage form performs as disclosed when the deutetrabenazine has a median particle size of 0.05 to 100 micron (μm), or 0.05 to 50 μm, or 1 μm to 30 μm, or 1 μm to 25 μm, or 5 μm to 30 μm, or 1 μm to 20 μm, or 5 μm to 25 μm, or 10 μm to 20 μm. The desired median particle size may be generated by, for example, milling the drug substance to micrometer sizes. In some embodiments, the deutetrabenazine has a particle size distribution characterized by a D₉₀ of not more than 15 μm. The D₉₀ is preferably not more than 14 μm, not more than 13 μm, not more than 12 μm, not more than 11 μm or not more than 10 μm. In some embodiments, the deutetrabenazine has a particle size distribution characterized by a D₁₀ of not more than 3 μm.

In some embodiments, the micronized deutetrabenazine is present in the first amount of micronized deutetrabenazine or the second amount of micronized deutetrabenazine or immediate release bead in a range of about 5 wt %-80 wt %, or 10 wt %-80 wt %, or 10 wt %-70 wt %, 20 wt %-60 wt %, 5 wt %-30 wt %, or 50 wt %-80 wt % of total weight of the dosage form. Deutetrabenazine may be present in the first amount of micronized deutetrabenazine or the second amount of micronized deutetrabenazine or immediate release bead in an amount of about (by wt %) 5.0, 6.0, 7.0, 8.0, 9.0, 10.0, 1.01, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 60.0, 61.0, 62.0, 63.0, 64.0, 65.0, 66.0, 67.0, 68.0, 69.0, 70.0, 71.0, 72.0, 73.0, 74.0, 75.0, 76.0, 77.0, 78.0, 79.0, 70.0, wt % of the weight of the core or of the sustained release beads, or in the immediate release bead, respectively.

The first or the second or the immediate release pharmaceutically acceptable excipient comprises, independently, an antioxidant, a binder, a filler, a surfactant, an anti-foaming agent or combinations thereof. Typically, more than one excipient is used for any one of the first or second dispersions with the first and/or second amount of the micronized deutetrabenazine, respectively. In some embodiments, the first or the second or the immediate release excipient comprises, independently, an antioxidant, which is a water-insoluble antioxidant. In some embodiments, the water-insoluble antioxidant is selected from the group consisting of propyl gallate, 6-ethoxy-1,2-digydro-2,2,4-trimethylquinoline (ethoxyquin), nordihydroguaiaretic acid (NDGA), butylated hydroxyanisole, butylated hydroxytoluene or any mixture thereof. In one specific embodiment, the antioxidant is selected from butylated hydroxytoluene (BHT), butylated hydroxyanisole (BHA), and combinations thereof. The antioxidant, preferably the water-insoluble antioxidant, is present in the dosage form in a range of 0.1 wt %-1.0 wt %, or about 0.2 wt %-1.0 wt %, or about 0.5 wt %-0.8 wt % of the weight of the first amount of micronized deutetrabenazine and the first pharmaceutically acceptable excipient or the second amount of micronized deutetrabenazine and the second pharmaceutically acceptable excipient or in the immediate release bead and may be present in an amount of (by wt %) 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0 wt % of the weight of the core, or the sustained release bead or in the immediate release bead, respectively.

The first and/or the second, and/or the immediate release excipient may, independently, comprise a binder. In some embodiments, the binder comprises a water-soluble binder, a water-insoluble binder or combinations thereof. In some embodiments, the binder comprises a water-soluble binder which may be a cellulose based binder including hydroxypropyl cellulose, and hydroxypropyl methylcellulose, polyvinyl pyrrolidone, polyvinyl alcohol, a polyacrylic acid polymer, polyether, carbohydrate polymer (natural or synthetic) or combinations thereof. In some embodiments, the binder is a cellulose-based binder selected from the group consisting of methyl cellulose (MC), ethyl cellulose (EC), propyl cellulose (PC), hydroxymethyl cellulose (HMC), hydroxyethyl cellulose (HEC), hydroxypropyl cellulose (HPC), hydroxypropyl methyl cellulose (HPMC), cellulose acetate and combinations thereof. In some embodiments, the binder is hydroxypropyl cellulose. In some embodiments, the binder is a polyether. Suitable polyethers include polyethylene glycol polymers. In further embodiments, the binder comprises a water-insoluble polymer, which comprises crospovidone, copovidone, microcrystalline cellulose, croscarmellose sodium, starch, sodium starch glycolate, colloidal silica, silica, ethyl cellulose, lactic acid polymer, a lactic acid and glutamic acid copolymer, polyvinyl acetate or combinations thereof. In some embodiments, the binder is present in the first amount of micronized deutetrabenazine and the first pharmaceutically acceptable excipient or the second amount of micronized deutetrabenazine and the second pharmaceutically acceptable excipient or in the immediate release bead in a range of 0.5 wt %-10.0 wt %, about 1.0 wt %-8.0 wt %, or about 2.0 wt %-6.0 wt % of the weight of the dosage form. The binder may present in the dosage form in an amount of (by wt. %) 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10.0 wt % of the weight of the core, or the sustained release bead, or in the immediate release bead, respectively.

In one embodiment, the weight ratio of the binder and the micronized deutetrabenazine in the first amount of micronized deutetrabenazine and/or the second amount of micronized deutetrabenazine, and/or the immediate release bead, independently, is about 5:1-1.5:1, or about 4.5:1-2:1 or about 4:1-2:1, or about 4:1 or about 2:1.

In some embodiments, the first and/or the second, and/or the immediate release excipient comprises, independently, a filler selected from the group consisting of a saccharide, a disaccharide, a polysaccharide, a polyalcohol, microcrystalline cellulose, natural and synthetic gums, gelatin, pregelatinized starch, polyvinylpyrrolidone, cellulose derivatives, dibasic calcium phosphate, kaolin, inorganic salts, calcium carbonate, sodium bicarbonate, sodium carbonate and combinations thereof. The saccharide may be for example, glucose, galactose, dextrose, fructose; a disaccharide may be for example, sucrose, lactose, lactose monohydrate, maltose, trehalose, maltose; a polysaccharide may be starch, maltodextrin; and a polyalcohol may be for example, sorbitol, xylitol, inositol, lactitol, mannitol, spray-dried mannitol. In some embodiments, the filler is microcrystalline cellulose, lactose monohydrate or a combination thereof. In some embodiments, the filler is lactose monohydrate, mannitol or a combination thereof. In some embodiments, the filler is present in the dosage form in a range of 5.0-50.0 wt %, 5.0-30.0 wt %, 10.0-40.0 wt %, or 10.0-40.0 wt %, of the weight of the first amount of micronized deutetrabenazine and the first pharmaceutically acceptable excipient or the second amount of micronized deutetrabenazine and the second pharmaceutically acceptable excipient or in the immediate release bead. In some embodiments, the excipient comprises about (by wt %) 5.0, 6.0, 7.0, 8.0, 9.0, 10.0, 1.01, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, or 50 wt % of the weight of the core or the sustained release bead, or in the immediate release bead, respectively.

In some embodiments of the dosage form, the first and/or the second, and/or the immediate release, pharmaceutically acceptable excipient comprises, independently, a surfactant. The surfactant may comprises sodium lauryl sulfate, sodium dodecyl sulfate, sodium laureth sulfate, docusate sodium, polysorbate, tween, polyoxyethylene 15 hydroxy stearate, polyoxyethylene castor oil derivatives, polyoxyethylene stearates, sorbitan fatty acid esters, polyoxyethylene alkyl ethers, polyoxyethylene nonylphenol ether or combinations thereof. In some embodiments, the surfactant is present in the first amount of micronized deutetrabenazine and the first pharmaceutically acceptable excipient or the second amount of micronized deutetrabenazine and the second pharmaceutically acceptable excipient or in the immediate release bead at a concentration of 2.0 wt %-12.0 wt % of the weight of the core or the sustained release bead, or the immediate release bead, respectively. The surfactant may present in the first amount of micronized deutetrabenazine and the first pharmaceutically acceptable excipient or the second amount of micronized deutetrabenazine and the second pharmaceutically acceptable excipient or in the immediate release bead in an amount of (by wt %), 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0. or 12.0 wt % of the weight of the core, or the sustained release bead, or in the immediate release bead, respectively.

In some embodiments, the first and/or the second, and/or the immediate release excipient comprises, independently, an anti-foaming agent, for example insoluble oils, polydimethylsiloxanes and other silicones, certain alcohols, stearates, glycols and combinations thereof. In various embodiments, the anti-foaming agent is simethicone, dimethicone, tilactase or peppermint oil. The ant-foaming agent may be simethicone 30% at up to about 2.0 wt % of the weight of the core, or the sustained release bead, or in the immediate release bead.

In some embodiments, an immediate release bead disclosed herein comprises an inert particle coated with micronized deutetrabenazine having a D₉₀ 10 to 15 micron (μm) and a pharmaceutically acceptable excipient comprising about 0.1 wt %-1.0 wt % of an antioxidant, about 0.5 wt %-10.0 wt % of a binder, about of 5.0 wt %-50.0 wt % of a filler, about 2.0 wt %-12.0 wt % of a surfactant, and about 0.3 wt %-3 wt % of an anti-foaming agent, based on the weight core or the immediate release bead.

In some embodiments, the sustained release beads comprise a first and/or a second pH-independent polymer coat. The pH-independent polymer coat may include ethylcellulose. In some embodiments, the pH-independent polymer coat includes a cellulose acetate, a mixture of cellulose acetates, ethylcellulose or a mixture of ethylcellulose and polyethylene glycol. In some embodiments, the pH-independent polymer coat comprises cellulose acetate. In some embodiments, the pH-independent polymer coat comprises a mixture of cellulose acetate NF 398-10 and cellulose acetate 320S. In yet other embodiments, the pH-independent polymer coat comprises a mixture of cellulose acetate and polyethylene glycol.

In certain embodiments, the sustained release beads comprise a first and/or a second pH-dependent polymer coat. In some embodiments, the sustained release bead comprises a first and/or a second pH-dependent polymer to target drug release at a pH 5-7.0 and targets the upper small intestine. The enteric polymer is methacrylic acid-ethyl acrylate copolymer. In some embodiments the sustained release bead comprises a first and/or a second pH-dependent polymer to target drug release at a pH>7.0 and targets the large intestine/colon. In some embodiments, the pH-dependent polymer coat that targets large intestine/colon comprises cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac and combinations thereof. In some embodiments, the pH-dependent polymer coat comprises methacrylic acid-methyl methacrylate copolymer. In some embodiments, the pH-dependent polymer coat comprises a mixture of cellulose acetate and polyethylene glycol. In some embodiments, the pH-dependent polymer coat comprises a mixture of ethyl cellulose and polyethylene glycol.

The first and/or a second pH-independent or first and/or a second pH-dependent polymer coat may further include a first and/or a second pharmaceutically acceptable plasticizer. The plasticizer may be triethyl citrate (TEC), triacetin, acetyl tributyl citrate, acetyl triethyl citrate, glycerin, a polyethylene glycol, polyethylene glycol monomethyl ether, propylene glycol, sorbitol sorbitan solution, castor oil, diacetylated monoglycerides, dibutyl sebacates, diethyl phthalate or combinations thereof. In some embodiments, the plasticizer comprises triethyl citrate.

In some embodiments of the dosage form, the first and/or a second pH-independent polymer coat or the first and/or a second pH-dependent polymer coat is present, independently, on the sustained release bead at a concentration of 15.0 wt %-50.0 wt %, or about 20.0 wt %-40.0 wt % of the weight of the sustained release bead.

Optionally, the sustained release beads further comprise a film coat, the film coat comprising a mixture of hydrophilic and hydrophobic polymers. In some embodiments, the hydrophilic polymer may be selected from polyacrylic acid, polyvinyl alcohol, polyethylene glycol, polyvinyl pyrrolidone, polyethylene oxide, alginic acid and its salts, chitosan, carrageenan, gum Arabic, guar gum, agar agar, gelatin, xanthan, locust bean gum, methyl cellulose, carboxymethyl cellulose, hydroxyethyl cellulose, hydroxypropyl cellulose, hydroxypropyl methylcellulose, starches, and combinations thereof. In some embodiments, the and hydrophobic polymer may be selected from ethyl cellulose, cellulose acetate, cellulose acetate phthalate, cellulose acetate butyrate, shellac, methacrylate and acrylate copolymers (enteric and non-enteric), poly(lactic acid), poly(lactide-co-glycolide), hydroxypropyl methylcellulose phthalate, hydroxypropyl methylcellulose acetate succinate, poly(vinyl acetate), and combinations thereof.

The dosage form may include a total of 6 mg-72 mg of micronized deutetrabenazine. In some embodiments, the dosage form comprises a total of 6 mg, or 12 mg, or 18 mg, or 24 mg, or 30 mg, or 36 mg, or 42 mg or 48 mg of micronized deutetrabenazine.

In some embodiments, the dosage form consists essentially of a population of sustained release beads comprising
a) a core comprising a first amount of micronized deutetrabenazine and a first pharmaceutically acceptable excipient; wherein the first pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate;
b) a first pH-independent polymer coat coating the core; and optionally further comprising
c) a capsule shell or pharmaceutical sachet packaging.

The core of the dosage form comprises a) immediate release granules, immediate release pellet or immediate release tablet comprising the first amount of micronized deutetrabenazine and the first pharmaceutically acceptable excipient or b) an inert particle coated with the first amount of micronized deutetrabenazine and the first pharmaceutically acceptable excipient.

In some embodiments, the first coat of pH-independent polymer comprises ethylcellulose, polyethylene glycol and triacetin, optionally further comprising povidone. In other embodiments, the first coat of pH-independent polymer coat comprises cellulose acetate and optionally polyethylene glycol (PEG). In some embodiments, the cellulose acetate comprises a mixture of cellulose acetate 398-10 and cellulose acetate 320S, optionally further comprising PEG 3350.

In some embodiments, the dosage form comprises at least one population of sustained release beads and one population of immediate release beads, wherein the immediate release beads comprise a) immediate release granules, immediate release pellet or immediate release tablet comprising the first amount of the micronized deutetrabenazine and the first pharmaceutically acceptable excipient or b) an inert particle coated with the first amount of the micronized deutetrabenazine and the first pharmaceutically acceptable excipient. In some embodiments, the first pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate. The sustained release beads comprise a core, which may consist essentially of the immediate release beads, further comprising a pH-dependent polymer coat that targets the small intestine. In some embodiments, the pH-dependent polymer coat comprises methacrylic acid and ethyl acrylate copolymer, and optionally triethyl citrate. In other embodiments, the sustained release beads comprise a core, which may consist essentially of the immediate release beads, further comprising a pH-dependent polymer coat that targets the large intestine/colon. In some embodiments, the pH-dependent polymer coat comprises methacrylic acid and methyl acrylate copolymer, and optionally triethyl citrate.

In some embodiments, the dosage forms of the disclosure comprise at least one population of sustained release beads and one population of immediate release beads.

In some embodiments, the dosage form comprises two populations of sustained release beads and one population of immediate release beads, one population of the sustained release beads targeting the small intestine and a second population of the sustained release beads targeting the large intestine/colon. The dosage form may be, for example, a capsule or a pharmaceutical sachet package.

Further provided herein, are methods useful in treating VMAT2 mediated disorders. In some embodiments, the method of treating a VMAT2 mediated disorder comprises orally administering to a patient in a need thereof, a controlled release dosage form as disclosed herein. The VMAT2 mediated disorder may be a hyperkinetic movement disorder. The hyperkinetic movement disorder may be a chronic disorder, for example dystonia, dyskinesia, Huntington's disease, tardive dyskinesia, and dyskinesia in cerebral palsy. In some embodiments, the method is effective in treating chorea associated with Huntington's disease. In some embodiments, the method is effective in treating tardive dyskinesia. The subjects afflicted with tardive dyskinesia may be concurrently administered an antipsychotic agent. In some embodiments, the method is effective in treating dyskinesia in cerebral palsy.

Further provided herein, are methods useful in treating VMAT2 mediated disorders. In some embodiments, the method of treating a VMAT2 mediated disorder comprises orally administering to a patient in a need thereof, a sustained release dosage form disclosed herein. The VMAT2 mediated disorder may be a hyperkinetic movement disorder. The hyperkinetic movement disorder may be a chronic disorder, for example dystonia, dyskinesia, Huntington's disease, tardive dyskinesia, and dyskinesia in cerebral palsy. In some embodiments, the method is effective in treating chorea associated with Huntington's disease. In some embodiments, the method is effective in treating tardive dyskinesia. The subjects afflicted with tardive dyskinesia may be concurrently administered an antipsychotic agent. In some embodiments, the method is effective in treating dyskinesia in cerebral palsy.

In certain embodiments, the multiparticulate dosage form according to any one of the embodiments disclosed herein, is administered with food.

In certain embodiments, the multiparticulate dosage form according to any one of the embodiments disclosed herein, is administered under fasting conditions.

The plasma profiles of the dosage form following administration are favorable. In one embodiment, a single dose administration of the once daily oral dosage form comprising 6 mg of micronized deutetrabenazine provides an in vivo plasma profile for total α- and β-dihydrodeutetrabenazine that includes a geometric mean AUC0-inf of about 90,000 to 142,750 h*pg/mL and/or a geometric mean Cmax of less than about 4,600 pg/mL.

In one embodiment, a single dose administration of the once daily oral dosage form comprising 12 mg of micronized deutetrabenazine provides an in vivo plasma profile for total α- and β-dihydrodeutetrabenazine that includes a geometric mean AUC0-inf of about 180,000 to 285,500 h*pg/mL and/or a geometric mean Cmax of less than about 9,200 pg/mL.

In one embodiment, a single dose administration of the once daily oral dosage form comprising 24 mg of micronized deutetrabenazine provides an in vivo plasma profile for total α- and β-dihydrodeutetrabenazine that includes a geometric mean AUC0-inf of about 360,000 to 571,000 h*pg/mL and/or a geometric mean Cmax of less than about 18,400 pg/mL.

In one embodiment, a single dose administration of the once daily oral dosage form comprising 36 mg of micronized deutetrabenazine provides an in vivo plasma profile for total α- and β-dihydrodeutetrabenazine that includes a geometric mean AUC0-inf of about 540,000 to 856,500 h*pg/mL and/or a geometric mean Cmax of less than about 27,600 pg/mL.

In one embodiment, a single dose administration of the once daily oral dosage form comprising 48 mg of micronized deutetrabenazine provides an in vivo plasma profile for total α- and β-dihydrodeutetrabenazine that includes a geometric mean AUC0-inf of about 720,000 to 1,142,000 h*pg/mL and/or a geometric mean Cmax of less than about 36,800 pg/mL.

In one embodiment, administration of the once daily oral dosage form comprising 6 mg of micronized deutetrabenazine provides an in vivo plasma profile for total α- and β-dihydrodeutetrabenazine that includes a geometric mean AUC0-24 of about 102,500 to 200,000 h*pg/mL at steady state and/or a mean Cmax of less than about 10,000 pg/mL at steady state.

In one embodiment, administration of the once daily oral dosage form comprising 12 mg of micronized deutetrabenazine provides an in vivo plasma profile for total α- and β-dihydrodeutetrabenazine that includes a geometric mean AUC0-24 of about 205,000 to 400,000 h*pg/mL at steady state and/or a mean Cmax of less than about 20,000 pg/mL at steady state.

In one embodiment, administration of the once daily oral dosage form comprising 24 mg of micronized deutetrabenazine provides an in vivo plasma profile for total α- and β-dihydrodeutetrabenazine that includes a geometric mean AUC0-24 of about 400,000 to 800,000 h*pg/mL at steady state and/or a mean Cmax of less than about 40,000 pg/mL at steady state.

In one embodiment, administration of the once daily oral dosage form comprising 36 mg of micronized deutetrabenazine provides an in vivo plasma profile for total α- and β-dihydrodeutetrabenazine that includes a geometric mean AUC0-24 of about 615,000 to 1,200,000 h*pg/mL at steady state and/or a mean Cmax of less than about 60,000 pg/mL at steady state.

In one embodiment, administration of the once daily oral dosage form comprising 48 mg of micronized deutetrabenazine provides an in vivo plasma profile for total α- and β-dihydrodeutetrabenazine that includes a geometric mean AUC0-24 of about 800,000 to 1,600,000 h*pg/mL at steady state and/or a mean Cmax of less than about 80,000 pg/mL at steady state.

In one embodiment, a single dose administration of the twice daily oral dosage form comprising 6 mg of micronized deutetrabenazine provides an in vivo plasma profile for total α- and β-dihydrodeutetrabenazine that includes a geometric mean AUC_{0-inf} of about 132±47 h*ng/ml and/or a geometric mean Cmax of less than about 15.5±3.5 ng/ml.

In one embodiment, a single dose administration of the twice daily oral dosage form comprising 12 mg of micronized deutetrabenazine provides an in vivo plasma profile for total α- and β-dihydrodeutetrabenazine that includes a geometric mean AUC_{0-inf} of about 289±115 h*ng/ml and/or a geometric mean Cmax of less than about 32.1±8.1 ng/mL.

In one embodiment, a single dose administration of the twice daily oral dosage form comprising 18 mg of micronized deutetrabenazine provides an in vivo plasma profile for total α- and β-dihydrodeutetrabenazine that includes a geometric mean AUC_{0-inf} of about 419±165 h*ng/mL and/or a geometric mean Cmax of less than about 47.8±12.0 ng/ml.

In one embodiment, a single dose administration of the twice daily oral dosage form comprising 24 mg of micronized deutetrabenazine provides an in vivo plasma profile for total α- and β-dihydrodeutetrabenazine that includes a geometric mean AUC_{0-inf} of about 580±229 h*ng/mL and/or a geometric mean Cmax of less than about 60.9±13.8 ng/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 6 mg of micronized deutetrabenazine, provides an in vivo plasma profile for total α- and β-dihydrodeutetrabenazine that includes a geometric mean AUC_{0-inf} of about 90,000 to 142,750 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 6 mg of micronized deutetrabenazine, provides an in vivo plasma profile for total α- and β-dihydrodeutetrabenazine that includes a geometric mean Cₘₐₓ of less than about 4,600 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 12 mg of micronized deutetrabenazine, provides an in vivo plasma profile for total α- and β-dihydrodeutetrabenazine that includes a geometric mean AUC_{0-inf} of about 180,00 to 285,500 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 12 mg of micronized deutetrabenazine, provides an in vivo plasma profile for total α- and β-dihydrodeutetrabenazine that includes a geometric mean Cₘₐₓ of less than about 9,200 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 24 mg of micronized deutetrabenazine, provides an in vivo plasma profile for total α- and β-dihydrodeutetrabenazine that includes a geometric mean AUC_{0-inf} of about 360,000 to 571,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 24 mg of micronized deutetrabenazine, provides an in vivo plasma profile for total α- and β-dihydrodeutetrabenazine that includes a geometric mean Cₘₐₓ of less than about 18,400 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 36 mg of micronized deutetrabenazine, provides an in vivo plasma profile for total α- and β-dihydrodeutetrabenazine that includes a geometric mean AUC_{0-inf} of about 540,000 to 856,500 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 36 mg of micronized deutetrabenazine, provides an in vivo plasma profile for total α- and β-dihydrodeutetrabenazine that includes a geometric mean Cₘₐₓ of less than about 27,600 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 48 mg of micronized deutetrabenazine, provides an in vivo plasma profile for total α- and β-dihydrodeutetrabenazine that includes a geometric mean AUC_{0-inf} of about 720,000 to 1,142,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 48 mg of micronized deutetrabenazine, provides an in vivo plasma profile for total α- and β-dihydrodeutetrabenazine that includes a geometric mean Cₘₐₓ of less than about 36,800 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 6 mg of micronized deutetrabenazine, provides an in vivo plasma profile for total α- and β-dihydrodeutetrabenazine at steady state that includes a mean AUC₀₋₂₄ of about 102,500 to 200,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 6 mg of micronized deutetrabenazine provides an in vivo plasma profile for total α- and β-dihydrodeutetrabenazine at steady state that includes a mean Cₘₐₓ of less than about 10,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 12 mg of micronized deutetrabenazine provides an in vivo plasma profile for total α- and β-dihydrodeutetrabenazine at steady state that includes a mean AUC₀₋₂₄ of about 205,000 to 400,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 12 mg of micronized deutetrabenazine, provides an in vivo plasma profile for total α- and β-dihydrodeutetrabenazine at steady state that includes a mean Cₘₐₓ of less than about 20,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 24 mg of micronized deutetrabenazine, provides an in vivo plasma profile for total α- and β-dihydrodeutetrabenazine at steady state that includes a mean AUC₀₋₂₄ of about 410,000 to 800,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 24 mg of micronized deutetrabenazine, provides an in vivo plasma profile for total α- and β-dihydrodeutetrabenazine at steady state that includes a mean Cₘₐₓ of less than about 40,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 36 mg of micronized deutetrabenazine, provides an in vivo plasma profile for total α- and β-dihydrodeutetrabenazine at steady state that includes a mean AUC₀₋₂₄ of about 615,000 to 1,200,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 36 mg of micronized deutetrabenazine, provides an in vivo plasma profile for total α- and β-dihydrodeutetrabenazine at steady state that includes a mean Cₘₐₓ of less than about 60,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 48 mg of micronized deutetrabenazine, provides an in vivo plasma profile for total α- and β-dihydrodeutetrabenazine at steady state that includes a mean AUC0-24 of about 820,000 to 1,600,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 48 mg of micronized deutetrabenazine, provides an in vivo plasma profile for total α- and β-dihydrodeutetrabenazine at steady state that includes a mean Cₘₐₓ of less than about 80,000 pg/mL.

In one embodiment, the invention provides a method of treating a hyperkinetic movement disorder in a subject in need thereof comprising orally administering to the subject a twice daily multiparticulate dosage form according to any one of the embodiments of the invention wherein single dose administration of the multiparticulate dosage form which comprises a total amount of 6 mg of micronized deutetrabenazine, provides an in vivo plasma profile for total α- and β-dihydrodeutetrabenazine that includes a mean Cₘₐₓ of less than about 15.5±3.5 ng/mL.

In one embodiment, the invention provides a method of treating a hyperkinetic movement disorder in a subject in need thereof comprising orally administering to the subject a twice daily multiparticulate dosage form according to any one of the embodiments of the invention wherein single dose administration of the multiparticulate dosage which comprises a total amount of 12 mg of micronized deutetrabenazine, provides an in vivo plasma profile for total α- and β-dihydrodeutetrabenazine that includes a mean Cₘₐₓ of less than about 32.1±8.1 ng/ml

In one embodiment, the invention provides a method of treating a hyperkinetic movement disorder in a subject in need thereof comprising orally administering to the subject a twice daily multiparticulate dosage form according to any one of the embodiments of the invention wherein single dose administration of the multiparticulate dosage which comprises a total amount of 18 mg of micronized deutetrabenazine, provides an in vivo plasma profile for total α- and β-dihydrodeutetrabenazine that includes a mean Cₘₐₓ of less than about 47.8±12.0 ng/ml

In one embodiment, the invention provides a method of treating a hyperkinetic movement disorder in a subject in need thereof comprising orally administering to the subject a twice daily multiparticulate dosage form according to any one of the embodiments of the invention wherein single dose administration of the multiparticulate dosage which comprises a total amount of 24 mg of micronized deutetrabenazine, provides an in vivo plasma profile for total α- and β-dihydrodeutetrabenazine that includes a mean Cₘₐₓ of less than about 60.9±13.8 ng/mL.

In some embodiments, about 50% of micronized deutetrabenazine is released within 7 hours, as measured in a USPIII dissolution device, pH 7.2. In some embodiments, about 30% of micronized deutetrabenazine is released within 2 hours, and about 65% is released within 6 hours and not less than (NLT) about 80% of micronized deutetrabenazine is released within 10 hours, as measured in a USPIII dissolution device, pH 7.2. In some embodiments, about 25% of micronized deutetrabenazine is released within 2 hours, and about 45% is released within 6 hours and NLT about 75% of micronized deutetrabenazine is released within 10 hours as measured in a USPIII dissolution device, pH 7.2. In some embodiments, about 15% of micronized deutetrabenazine is released within 2 hours, about 35% is released within 6 hours and NLT about 55% of micronized deutetrabenazine is released within 10 hours as measured in a USPIII dissolution device, pH 7.2.

In some embodiments, about 50% of micronized deutetrabenazine is released within 4 hours, as measured in a USPIII dissolution device, pH 7.2. In some embodiments not less than (NLT) about 80% of micronized deutetrabenazine is released within 8 hours, as measured in a USPIII dissolution device, pH 7.2.

Further provided herein is a process for manufacturing the immediate release beads or the core of the sustained release beads, comprising the steps of:
a) providing a dispersion of a first amount of micronized deutetrabenazine with a first pharmaceutically acceptable excipient, wherein the first pharmaceutically acceptable excipient comprises: an antioxidant, a binder, an anti-foaming agent, a filler, and a surfactant;
b) forming immediate release granules, immediate release pellet or immediate release tablet from the dispersion of a); or coating an inert particle with the dispersion of a);
thereby generating the immediate release beads or the core of the sustained release beads, respectively.

Further provided is a process for manufacturing the sustained release beads comprising the steps of:
a) providing a core, wherein the core comprises immediate release granules, immediate release pellet or immediate release tablet comprising a dispersion of a first amount of micronized deutetrabenazine and a first pharmaceutically acceptable excipient; or an inert particle coated with a dispersion of a first amount of micronized deutetrabenazine and a first pharmaceutically acceptable excipient;
b) coating the core of a) with a first coat selected from pH-independent polymer coating, a pH-dependent polymer coating or with a pH-independent polymer coating and a pH-dependent polymer coating;
thereby generating sustained release beads.

In some embodiments, the process further comprises:
c) coating the first coat with a second amount of micronized deutetrabenazine and a second pharmaceutically acceptable excipient, wherein the second pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate or mixtures thereof;
thereby generating sustained release beads comprising a second amount of immediate release deutetrabenazine and a second pharmaceutically acceptable excipient.

In some embodiments, the process further comprises:
d) coating the sustained release beads comprising a second amount of immediate release deutetrabenazine and a second pharmaceutically acceptable excipient, with a second coat selected from pH-independent polymer, a pH-dependent polymer coating or with a pH-independent polymer coating and a pH-dependent polymer;
thereby generating sustained release beads comprising a second coat.

In some embodiments, the process further comprises, subsequent to any one of steps b-d, coating with a film coat, the film coat comprising a mixture of hydrophilic and hydrophobic polymers.

In some embodiments of the core or immediate release particles, the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate.

The dosage form may be manufactured by loading a capsule shell or a sachet with a population of sustained release beads comprising a core and a pH-independent coating.

The dosage form may be manufactured by loading a capsule shell or a sachet with a population of immediate release beads and a population of sustained release beads comprising a core and a pH-dependent coating, the pH-dependent coating targeting the small intestine.

The dosage form may be manufactured by loading a capsule shell or a sachet with a population of immediate release beads and a population of sustained release beads comprising a core and a pH-dependent coating, the pH-dependent coating targeting the large intestine/colon.

The dosage form may be manufactured by loading a capsule shell or a sachet with a population of immediate release beads, a population of sustained release beads comprising a core and a pH-dependent coating targeting the small intestine and a population of sustained release beads comprising a core and a pH-dependent coating targeting the large intestine/colon.

The dosage form may be manufactured by loading a capsule shell or a sachet with a population of sustained release beads comprising a core, a pH-independent coating, a second immediate release coat and a second pH-independent coating.

The dosage form may be manufactured by loading a capsule shell or a sachet with a population of immediate release beads and sustained release beads comprising a core, a pH-independent coating, a second immediate release coat and a second pH-independent coating.

The dosage form may be manufactured by loading a capsule shell or a sachet with a population of sustained release beads comprising a core, a pH-dependent coating, a second immediate release coat and a second pH-dependent coating.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the disclosure.

### Example 1—Manufacturing Process Development

The manufacturing process for the multiparticulate dosage form includes the following steps:
a. Manufacturing of micronized deutetrabenazine dispersion
b. Coating of particles with micronized deutetrabenazine dispersion to generate micronized deutetrabenazine coated particles or manufacture of core granules/pellets/tablets from micronized deutetrabenazine dispersion;
c. Sustained release coating of the micronized deutetrabenazine particles;
d. Optional packaging/encapsulation

### 1. Drug Substance Physical Characterization

The deutetrabenazine particle size distributions following manufacture (untreated), micronization (air jet mill) are shown in Table 1.

TABLE 1
Particle Size Distribution (PSD) of
unmilled and milled drug substance

| PSD | unmilled | micro-milled |
|---|---|---|
| D₁₀ (μm) | 9.08 | 1.08 |
| D₅₀ (μm) | 59.66 | 3.31 |
| D₉₀ (μm) | 213.07 | 7.05 |

Using a Mastersizer 3000 (Malvern Instruments), the following settings were used for dry measurement of the micro-milled and unmilled deutetrabenazine:

| Analysis model | Mie |
|---|---|
| Obscuration | 1.12% |
| Sample measurement time | 24 sec |

The dosage forms disclosed herein were developed to achieve similar pharmacokinetics (PK) of 2 doses of AUSTEDO® 12 mg tablets with either a single daily dose (QD) or a twice daily dose (BID).

### Example 2: Milling of Deutetrabenazine to a Micro Size

Deutetrabenazine particle size was reduced to a micro size (<10 micron) using quadro mill and micronized using an air jet mill.

### Example 3: Immediate Release and Sustained Release (pH Dependent) Dosage Form

Following milling, a solution of lactose, mannitol or mixture thereof is prepared and added to the micronized active material and mixed for 30 minutes using an air mixer. The resultant deutetrabenazine dispersion is sprayed on microcrystalline cellulose spheres using a Glatt fluid bed coater to generate deutetrabenazine-coated particles. A first portion of the deutetrabenazine coated particles is left as is (i.e. immediate release population); a second portion is further coated with a sustained release coating (methacrylic acid and ethyl acrylate copolymer dispersion, pH5.5-7); and a third portion is coated with a second sustained release coating (methacrylic acid and methyl methacrylate copolymer dispersionm pH>7). The sustained release particles are further coated with a mixture of guar gum and ethylcellulose

The immediate release particles and the two populations of the sustained release particles are filled into a capsule shell. Dissolution of the filled capsule is performed in a USPIII apparatus at 10 dpm. The pH values in the apparatus are selected based on the pH of the GI. A pH gradient was 0-1 hr in 0.1N HCl, 1 hr-3 hrs in phosphate buffer pH 6.8 and 3 hrs-6 hrs in phosphate buffer pH 7.2. Samples are collected at 1, 2, 3, 4, 5 and 6 hrs time points.

### Example 4: Immediate Release and Sustained Release (pH Independent) Dosage Form

Following milling, a solution of lactose, mannitol or mixture thereof is prepared and added to the micronized active material and mixed for 30 minutes using an air mixer. The resultant deutetrabenazine dispersion is sprayed on microcrystalline cellulose spheres using a Glatt fluid bed coater to generate deutetrabenazine-coated particles. A first portion of the deutetrabenazine coated particles is left as is (i.e. immediate release population); a second portion is further coated with a sustained release coating (a mixture of cellulose acetates). The sustained release particles are further coated with a mixture of guar gum and ethylcellulose

The immediate release particles and the sustained release particles are filled into a capsule shell. Dissolution of the filled capsule is performed in a USPIII apparatus at 10 dpm. The pH values in the apparatus are selected based on the pH of the GI. A pH gradient was 0-1 hr in 0.1N HCl, 1 hr-3 hrs in phosphate buffer pH 6.8 and 3 hrs-6 hrs in phosphate buffer pH 7.2. Samples are collected at 1, 2, 3, 4, 5 and 6 hrs time points.

### Example 5: Sustained Release Dosage Form

Following milling, a solution of lactose, mannitol or mixture thereof is prepared and added to the micronized active material and mixed for 30 minutes using an air mixer. The resultant deutetrabenazine dispersion is sprayed on microcrystalline cellulose spheres using a Glatt fluid bed coater to generate deutetrabenazine-coated particles. The deutetrabenazine-coated particles are further coated with a sustained release coating (a mixture of cellulose acetates). The sustained release particles are further coated with a second deutetrabenazine dispersion. The sustained release particles are further coated with a second sustained release coating (a mixture of cellulose acetates). The sustained release particles are further coated with a mixture of guar gum and ethylcellulose.

The sustained release particles are filled into a capsule shell. Dissolution of the filled capsule is performed in a USPIII apparatus at 10 dpm. The pH values in the apparatus are selected based on the pH of the GI. A pH gradient was 0-1 hr in 0.1N HCl, 1 hr-3 hrs in phosphate buffer pH 6.8 and 3 hrs-6 hrs in phosphate buffer pH 7.2. Samples are collected at 1, 2, 3, 4, 5 and 6 hrs time points.

### Example 6—Single Dose Bioavailability Assessment

Microparticulate dosage forms containing deutetrabenazine are produced as disclosed in Example 1 and studied in a single dose pharmacokinetic study.

The primary objective is to assess the comparative bioavailability (BA) of deutetrabenazine and deuterated α- and β-dihydrotetrabenazine (deuHTBZ) metabolites following a single administration of the once daily microparticulate dosage form (Test) compared to a single 12 mg Austedo® tablet administered twice, 12 hours apart (bid), under fasted conditions.

Study Population and Number of Subjects: The study includes healthy male and female non-smoking subjects.

Duration of Subject Participation: The study includes a screening period of 2-4 weeks (period 1), an open label treatment period with the test dosage forms (Test) and the reference formulation (Ref) (period 2), and a follow-up visit at least 1 day later (period 3).

### Treatments:

### Treatment Sequence A:

Day 1—administration of Test.

Days 2-3—at least 6 hours wash out of Test followed by administration of Ref.

### Treatment Sequence B:

Day 1—administration of Ref

Days 2-3—at least 6 hours wash out of Ref, followed by administration of Test.

The primary objective was addressed using the following parameters:
maximum observed concentration (Cmax)
area under the plasma concentration-time (AUC) from time 0 to the time of the last measurable plasma concentration (AUC0-t)
AUC extrapolated to infinity (AUC0-∞)
AUC from time 0 to 24 hours post dose (AUC0-24h)

### Analyses

AUC0-t, AUC0-∞, and AUC0-24h are calculated using the trapezoidal rule. The Cmax, AUC0-t, AUC0-∞, and AUC0-24h data are natural log-transformed prior to the statistical analysis. Comparisons of Cmax, AUC0-t, AUC0-∞, and AUC0-24h between treatments (T2A vs R) will be carried out using a separate parametric analysis of variance (ANOVA) model with fixed effect terms for sequence, period, treatment group, and a random effect of subject within sequence. The difference between the reference formulation (Ref) and the test formulation (Test) will be evaluated by constructing 90% confidence intervals for the Test/Ref ratios, based on the least-square means from the ANOVA for the log-transformed Cmax, AUC0-t, AUC0-∞ and AUC0-24h. The treatment difference and the associated 90% confidence interval estimated from the ANOVA on the log scale will be back-transformed to obtain the estimated ratio of geometric means between treatment groups and the 90% confidence interval for this ratio.

### Results

The once-daily dose of Test dosage forms provide similar deuHTBZ plasma concentrations observed for the Ref. The multiparticulate dosage forms disclosed herein are administered once daily and provide a similar treatment effect to that of AUSTEDO and also have no safety concerns.

### Example 6A—Single Dose Bioavailability Assessment

Microparticulate dosage forms containing deutetrabenazine are produced as disclosed in Examples 4 and/or 5 and studied in a single dose pharmacokinetic study.

The primary objective is to assess the comparative bioavailability (BA) of deutetrabenazine and deuterated α- and β-dihydrotetrabenazine (deuHTBZ) metabolites following a single administration of the 12 mg microparticulate dosage form administered twice, 12 hours apart (bid) (Test) compared to a single 12 mg Austedo® tablet administered twice, 12 hours apart (bid), under fasted conditions.

Study Population and Number of Subjects: The study includes healthy male and female non-smoking subjects.

Duration of Subject Participation: The study includes a screening period of 2-4 weeks (period 1), an open label treatment period with the test dosage forms (Test) and the reference formulation (Ref) (period 2), and a follow-up visit at least 1 day later (period 3).

### Treatments:

### Treatment Sequence A:

Day 1—administration of Test.

Days 2-3—at least 6 hours wash out of Test followed by administration of Ref.

Treatment sequence B:

### Day 1—Administration of Ref

Days 2-3—at least 6 hours wash out of Ref, followed by administration of Test.

The primary objective was addressed using the following parameters:
maximum observed concentration (Cmax)
area under the plasma concentration-time (AUC) from time 0 to the time of the last measurable plasma concentration (AUC0-t)
AUC extrapolated to infinity (AUC0-∞)
AUC from time 0 to 24 hours post dose (AUC0-24h)

### Analyses

AUC0-t, AUC0-∞, and AUC0-24h are calculated using the trapezoidal rule. The Cmax, AUC0-t, AUC0-∞, and AUC0-24h data are natural log-transformed prior to the statistical analysis. Comparisons of Cmax, AUC0-t, AUC0-∞, and AUC0-24h between treatments (T2A vs R) will be carried out using a separate parametric analysis of variance (ANOVA) model with fixed effect terms for sequence, period, treatment group, and a random effect of subject within sequence. The difference between the reference formulation (Ref) and the test formulation (Test) will be evaluated by constructing 90% confidence intervals for the Test/Ref ratios, based on the least-square means from the ANOVA for the log-transformed Cmax, AUC0-t, AUC0-∞ and AUC0-24h. The treatment difference and the associated 90% confidence interval estimated from the ANOVA on the log scale will be back-transformed to obtain the estimated ratio of geometric means between treatment groups and the 90% confidence interval for this ratio.

### Results

The twice-daily dose of Test dosage forms provide similar deuHTBZ plasma concentrations observed for the Ref. The multiparticulate dosage forms disclosed herein are administered twice daily and provide a similar treatment effect to that of AUSTEDO and also have no safety concerns.

### Example 7—Multiple Dose Bioavailability Assessment

The multiparticulate dosage forms containing 24 mg of deutetrabenazine were produced as disclosed in Example 1 and are studied in an open label, randomized, multiple-dose, 2-way crossover study in healthy volunteers.

The primary objective is to assess the bioequivalence (BE) of administration of Test, once daily (qd) compared to bid administration of Ref, under fasted or fed conditions.

Treatment includes 7 days repeated dosing of Test once daily versus 7 days repeated dosing of Ref, bid.

AUCt, Cₘₐₓ, tₘₐₓ, Cₘᵢₙ, Cₐᵥ for deutetrabenazine and deuHTBZ are analyzed, at steady state.

### Results

Multiple dosing of Test has comparable PK parameters to that of Ref, at steady state. Therefore similar efficacy response is expected with once daily administration, having no safety concerns.

### Example 7A—Multiple Dose Bioavailability Assessment

The multiparticulate dosage forms containing 12 mg of deutetrabenazine were produced as disclosed in Example 4 and/or 5 and are studied in an open label, randomized, multiple-dose, 2-way crossover study in healthy volunteers.

The primary objective is to assess the bioequivalence (BE) of administration of Test, twice daily (bid) compared to bid administration of Ref, under fasted or fed conditions.

Treatment includes 7 days repeated dosing of Test once daily versus 7 days repeated dosing of Ref, bid.

AUCt, Cₘₐₓ, tₘₐₓ, Cₘᵢₙ, Cₐᵥ for deutetrabenazine and deuHTBZ are analyzed, at steady state.

### Results

Multiple dosing of Test has comparable PK parameters to that of Ref, at steady state. Therefore similar efficacy response is expected with twice daily administration, having no safety concerns.

### Example 8: Food Effect Study

Multiparticulate dosage forms containing 24 mg deutetrabenazine are produced as disclosed in Example 1 and studied in an open label, randomized, two-way crossover study, to assess the comparative bioavailability of deutetrabenazine and deuHTBZ in the fed compared to the fasted state, following a single administration of 24 mg, once daily (qd) multiparticulate formulation.

### Treatment Includes:

A—24 mg, once daily (qd) multiparticulate formulation given as a single oral dose with water after an overnight fast of at least 10 hours.

B—24 mg, once daily (qd) multiparticulate formulation given as a single oral dose with water, 30 minutes after the start of standardized high calorie, high fat breakfast administered after an overnight fast of at least 10 hours.

Subject will receive treatments A/B with at least 6 days washout period.

AUCt, Cmax, tmax, Cmin, Cav for deutetrabenazine and deuHTBZ will be analyzed.

### Results

The similar plasma concentrations of deutetrabenazine and deuHTBZ, following single administration with or without food, show that the multiparticulate dosage from can be administered regardless of food.

### Example 8A: Food Effect Study

Multiparticulate dosage forms containing 12 mg deutetrabenazine are produced as disclosed in Example 4 and/or 5 and studied in an open label, randomized, two-way crossover study, to assess the comparative bioavailability of deutetrabenazine and deuHTBZ in the fed compared to the fasted state, following a single administration of 12 mgtwice, once daily (bid) multiparticulate formulation.

### Treatment Includes:

A—12 mg, twice daily (bid) multiparticulate formulation given with water after an overnight fast of at least 10 hours.

B—12 mg, twice daily (bid) multiparticulate formulation given with water, 30 minutes after the start of standardized high calorie, high fat breakfast administered after an overnight fast of at least 10 hours.

Subject will receive treatments A/B with at least 6 days washout period.

AUCt, Cmax, tmax, Cmin, Cav for deutetrabenazine and deuHTBZ will be analyzed.

### Results

The similar plasma concentrations of deutetrabenazine and deuHTBZ, following administration with or without food, show that the multiparticulate dosage from can be administered regardless of food.

### Example 9: In Vitro Dissolution Study in Alcoholic Solutions

Multiparticulate dosage forms containing 24 mg deutetrabenazine are produced as disclosed in Example 1 and tested for drug release, to evaluate the dissolution similarities in 0.1 N HCl medium and 0.1 N HCl+5% ethanol, 0.1 N HCl+10% ethanol, 0.1 N HCl+20% ethanol and 0.1 N HCl+40% ethanol media.

### Results

There is no difference in drug release in 0%-40% alcohol media for the dosage forms of the present invention. Dosages forms containing 24 mg deutetrabenazine are produced as disclosed in Example 1, exhibit sustained release of deutetrabenazine without initial dose dumping and maintains the drug release over a period of more than 8 hours.

All patents, patent applications, and publications mentioned in the specification are indicative of the levels of those of ordinary skill in the art to which the invention pertains. All patents, patent applications, and publications are herein incorporated by reference to the same extent as if each individual publication was specifically and individually indicated to be incorporated by reference. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

### Aspects

1. A controlled release oral dosage form for once daily administration of deutetrabenazine comprising a population of sustained release beads; wherein the sustained release beads comprise
a core comprising
   a first amount of micronized deutetrabenazine and a first pharmaceutically acceptable excipient, and
   further comprising a first coat selected from a pH-independent polymer coat, a pH-dependent polymer coat, or a pH-independent polymer coat further coated with a pH-dependent polymer coat.

2. The dosage form of Aspect 1, wherein the core comprises
a) immediate release granules, immediate release pellet or immediate release tablet comprising the first amount of micronized deutetrabenazine and the first pharmaceutically acceptable excipient; or
b) an inert particle coated with the first amount of the micronized deutetrabenazine and the first pharmaceutically acceptable excipient.

3. The dosage form of Aspect 1 or 2, wherein the sustained release beads are further coated with a second amount of micronized deutetrabenazine and a second pharmaceutically acceptable excipient on top of the first coat.

4. The dosage form of Aspect 3, wherein the sustained release beads are further coated with a second coat selected from a pH-independent polymer coat, a pH-dependent polymer coat or a pH-independent polymer coat further coated with a pH-dependent polymer coat, on top of the second amount of the micronized deutetrabenazine and the second pharmaceutically acceptable excipient.

5. The dosage form of any one of Aspects 1-4, further comprising a population of immediate release beads; wherein the population of immediate release beads comprises
a) immediate release granules, immediate release pellets or immediate release tablets comprising an immediate release amount of micronized deutetrabenazine and immediate release pharmaceutically acceptable excipient; or
b) an inert particle coated with an immediate release amount of micronized deutetrabenazine and an immediate release pharmaceutically acceptable excipient.

6. The dosage form of any one of Aspects 1-5, wherein the first amount of micronized deutetrabenazine and/or the pharmaceutically acceptable excipient and the second amount of micronized deutetrabenazine and/or the pharmaceutically acceptable excipient and the immediate release amount of micronized deutetrabenazine and/or immediate release pharmaceutically acceptable excipient are identical, or wherein the first amount of deutetrabenazine and/or the pharmaceutically acceptable excipient and the second amount of micronized deutetrabenazine and/or the pharmaceutically acceptable excipient and the immediate release amount of micronized deutetrabenazine and/or immediate release pharmaceutically acceptable excipient are different.

7. The dosage form of any one of Aspects 1-6, wherein the pharmaceutically acceptable excipient in the first amount of micronized deutetrabenazine and the first pharmaceutically acceptable excipient and the second amount of micronized deutetrabenazine and the second pharmaceutically acceptable excipient and the immediate release amount of micronized deutetrabenazine and immediate release pharmaceutically acceptable excipient, are identical or
wherein the pharmaceutically acceptable excipient in the first amount of micronized deutetrabenazine and the first pharmaceutically acceptable excipient and the second amount of micronized deutetrabenazine and the second pharmaceutically acceptable excipient and the immediate release amount of micronized deutetrabenazine and immediate release pharmaceutically acceptable excipient, are different.

8. The dosage form of any one of Aspects 1-7, wherein the micronized deutetrabenazine has a median particle size of 0.05 to 100 micron, or 1 to 30 micron, or 5 to 25 micron.

9. The dosage form of Aspect 8, wherein the micronized deutetrabenazine has a particle size distribution characterized by a D₉₀ of about 10 to about 15 micron.

10. The dosage form of Aspect 8, wherein the micronized deutetrabenazine has a particle size distribution characterized by a D₅₀ of about 10 to about 20 micron.

11. The dosage form of Aspect 8, wherein the micronized deutetrabenazine has a particle size distribution characterized by a D₁₀ of not more than 3 micron.

12. The dosage form of Aspect 8, wherein the micronized deutetrabenazine has a particle size distribution characterized by a D₉₀ of not more than 15 micron, a D₅₀ of about 10 to about 20 micron and a D₁₀ of not more than 3 micron.

13. The dosage form of any one of Aspects 1-12, wherein the micronized deutetrabenazine is present, independently, in the first amount of micronized deutetrabenazine or in the second amount of micronized deutetrabenazine or in the immediate release amount at a concentration of 5 wt %-80 wt %, or 10 wt %-80 wt %, or 10 wt %-70 wt %, 20 wt %-60 wt %, 5 wt %-30 wt %, or 50 wt %-80 wt % of weight of the core of the sustained release bead or of the immediate release bead.

14. The dosage form of any one of Aspects 1-13, wherein the first pharmaceutically acceptable excipient or the second pharmaceutically acceptable excipient or the immediate release pharmaceutically acceptable excipient, independently comprises any one of an antioxidant, a binder, a filler, a surfactant, an anti-foaming agent or a combination thereof.

15. The dosage form of Aspect 14, wherein the first pharmaceutically acceptable excipient or the second pharmaceutically acceptable excipient or the immediate release pharmaceutically acceptable excipient, independently comprises an antioxidant, which is preferably a water-insoluble antioxidant.

16. The dosage form of Aspect 15, wherein the water-insoluble antioxidant is selected from the group consisting of butylated hydroxytoluene (BHT), butylated hydroxyanisole (BHA), propyl gallate, 6-ethoxy-1,2-digydro-2,2,4-trimethylquinoline (ethoxyquin), nordihydroguaiaretic acid (NDGA), sodium metabisulfite (SMB), a tocopherol and combinations thereof.

17. The dosage form of Aspect 16, wherein the water-insoluble antioxidant comprises butylated hydroxytoluene (BHT), butylated hydroxyanisole (BHA) or a combination thereof.

18. The dosage form of any one of Aspects 15-17, wherein the first pharmaceutically acceptable excipient or the second pharmaceutically acceptable excipient or the immediate release pharmaceutically acceptable excipient, independently comprise a water-insoluble antioxidant at a concentration of 0.1 wt %-1.0 wt % of the weight of the core or of the sustained release bead or of the immediate release bead.

19. The dosage form of any one of Aspects 14-18, wherein the first, second or immediate release pharmaceutically acceptable excipient comprises a binder.

20. The dosage form of Aspect 19, wherein the binder comprises a water-soluble binder, a water-insoluble binder or combinations thereof.

21. The dosage form of Aspect 20, wherein the binder is a water-soluble binder which comprises hydroxypropyl cellulose, hydroxypropyl methylcellulose, polyvinyl pyrrolidone, polyvinyl alcohol, a polyacrylic acid polymer, polyether, natural or synthetic carbohydrate polymer, or combinations thereof.

22. The dosage form of Aspect 19 or Aspect 20, wherein the binder comprises a water-insoluble polymer which is crospovidone, copovidone, microcrystalline cellulose, croscarmellose sodium, starch, sodium starch glycolate, colloidal silica, silica, ethyl cellulose, lactic acid polymer, a lactic acid and glutamic acid copolymer, polyvinyl acetate or combinations thereof.

23. The dosage form of Aspect 20 or Aspect 21, wherein the binder comprises a polyether, preferably a PEG.

24. The dosage form of any one of Aspects 19-23, wherein the first pharmaceutically acceptable excipient or the second pharmaceutically acceptable excipient or the immediate release pharmaceutically acceptable excipient comprise, independently, the binder at a concentration of 0.5 wt %-10.0 wt % of the weight of the core or of the sustained release bead or of the immediate release bead.

25. The dosage form of any one of Aspects 14-24, wherein the first, second or immediate release pharmaceutically acceptable excipient comprises a filler selected from the group consisting of a saccharide, a disaccharide, a polysaccharide, a polyalcohol, microcrystalline cellulose, natural and synthetic gums, pregelatinized starch, polyvinylpyrrolidone, cellulose derivatives, dibasic calcium phosphate, kaolin, inorganic salts, calcium carbonate, sodium bicarbonate, sodium carbonate, and combinations thereof.

26. The dosage form of Aspect 25, wherein the filler comprises microcrystalline cellulose, a saccharide, a polyalcohol or a combination thereof;
wherein the saccharide preferably comprises lactose monohydrate and wherein the polyalcohol preferably comprises mannitol.

27. The dosage form of Aspect 25 or Aspect 26, wherein the first pharmaceutically acceptable excipient or the second pharmaceutically acceptable excipient or the immediate release pharmaceutically acceptable excipient comprise, independently, the filler at a concentration of 5.0-50.0 wt % of the weight of the core or of the sustained release bead or of the immediate release bead.

28. The dosage form of any one of Aspects 14-27, wherein the first, second or immediate release pharmaceutically acceptable excipient comprises a surfactant.

29. The dosage form of Aspect 28, wherein the surfactant comprises sodium lauryl sulfate, sodium dodecyl sulfate, sodium laureth sulfate, docusate sodium, polysorbate, tween, polyoxyethylene 15 hydroxy stearate, polyoxyethylene castor oil derivatives, polyoxyethylene stearates, sorbitan fatty acid esters, polyoxyethylene alkyl ethers, polyoxyethylene nonylphenol ether or combinations thereof.

30. The dosage form of Aspect 28 or Aspect 29, where in the first pharmaceutically acceptable excipient the second pharmaceutically acceptable excipient or the immediate release pharmaceutically acceptable excipient comprise, independently, the surfactant at a concentration of 2.0 wt %-12.0 wt % of the weight of the core or of the sustained release bead or of the immediate release bead.

31. The dosage form of any one of Aspects 14-30, wherein the first, second or immediate release pharmaceutically acceptable excipient comprises an anti-foaming agent.

32. The dosage form of Aspect 31, wherein the anti-foaming agent comprises insoluble oils, polydimethylsiloxanes and other silicones, certain alcohols, stearates, glycols and combinations thereof, preferably simethicone, dimethicone, tilactase or peppermint oil.

33. The dosage form of Aspect 31 or Aspect 32, wherein the first pharmaceutically acceptable excipient the second pharmaceutically acceptable excipient or the immediate release pharmaceutically acceptable excipient, independently comprise the anti-foaming, at a concentration of 0.3 wt %-3.0 wt % of the weight of the core or of the sustained release bead or of the immediate release bead.

34. The dosage form of any one of the preceding Aspects, wherein the sustained release bead comprises a first and optionally a second pH-independent polymer coat coating the core.

35. The dosage form of Aspect 34, wherein the first and/or the second pH-independent polymer coat comprises, independently, a cellulose acetate, a mixture of cellulose acetates, cellulose acetate and polyethylene glycol, ethylcellulose, or a mixture of ethylcellulose and polyethylene glycol.

36. The dosage form of Aspect 35, wherein the first and/or the second pH-independent polymer coat comprises cellulose acetate.

37. The dosage form of Aspect 36, wherein the first and/or the second pH-independent polymer coat comprises a mixture of cellulose acetate NF 398-10 and cellulose acetate 320S.

38. The dosage form of any one of Aspects 34-37, wherein the pH-independent polymer coat comprises a mixture of ethylcellulose and polyethylene glycol.

39. The dosage form of any one of Aspects 1-33, wherein the sustained release beads comprise a first and optionally a second pH-dependent polymer coat coating the core.

40. The dosage form of any one of Aspects 34-38, wherein the sustained release beads comprise a first and/or a second pH-dependent polymer coat coating the first and/or the second pH-independent polymer coat.

41. The dosage form of Aspect 39 or Aspect 40, wherein the first and/or the second pH-dependent polymer coat is formulated to dissolve at a pH of about 5.0-7.0.

42. The dosage form of Aspect 41, wherein the first and/or the second pH-dependent polymer coat comprises methacrylic acid-ethyl acrylate copolymer, hydroxypropylmethyl cellulose phthalate (HPMCP), alginates, carboxymethylcellulose, or a combination thereof.

43. The dosage form of Aspect 42, wherein the first and/or the second pH-dependent polymer coat comprises methacrylic acid-ethyl acrylate copolymer.

44. The dosage form of Aspect 39 or Aspect 40, wherein the first and/or the second pH-dependent polymer coat is formulated to dissolve at a pH above 7.0.

45. The dosage form of Aspect 44, wherein the first and/or the second pH-dependent polymer coat comprises cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac, or combinations thereof.

46. The dosage form of Aspect 45, wherein the first and/or the second pH-dependent polymer coat comprises methacrylic acid-methyl methacrylate copolymer.

47. The dosage form of any one of Aspects 34-46, wherein the first and/or the second pH-independent polymer coat or the first and/or the second pH-dependent polymer coat further comprises a pharmaceutically acceptable plasticizer.

48. The dosage form of Aspect 47, wherein the plasticizer comprises triethyl citrate (TEC), triacetin, acetyl tributyl citrate, acetyl triethyl citrate, glycerin, a polyethylene glycol, polyethylene glycol monomethyl ether, propylene glycol, sorbitol sorbitan solution, castor oil, diacetylated monoglycerides, dibutyl sebacates, diethyl phthalate or combinations thereof.

49. The dosage form of Aspect 48, wherein the plasticizer comprises triethyl citrate.

50. The dosage form of any one of Aspects 34-49, wherein the first and/or the second pH-independent polymer coat or the first and/or the second pH-dependent polymer coat is present in the sustained release bead at a concentration of 15.0 wt %-50.0 wt % of the weight of the sustained release bead.

51. The dosage form of any one of Aspects 34-49, wherein the pH-independent polymer coat or the pH-dependent polymer coat is present on the sustained release bead at a concentration of 20.0 wt %-40.0 wt % of the weight of the sustained release bead.

52. The dosage form of any one of the preceding Aspects, wherein the dosage form comprises a total of 6 mg-72 mg of micronized deutetrabenazine.

53. The dosage form of Aspect 52, wherein the dosage form comprises a total of 6 mg, or 12 mg, or 18 mg, or 24 mg, or 30 mg, or 36 mg, or 42 mg or 48 mg of micronized deutetrabenazine.

54. The dosage form of any one of Aspects 1˜4 or 8-53, consisting essentially of a population of sustained release beads.

55. The dosage form of Aspect 54 wherein the population of sustained release beads comprises:
a) a core comprising a first amount of micronized deutetrabenazine and a first pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate;
b) a first pH-independent polymer coat coating the core; and optionally further comprising
c) a second amount of micronized deutetrabenazine and a second pharmaceutically acceptable excipient coat coating the first pH-independent polymer coat; and optionally further comprising
d) a second pH-independent polymer coat coating the second amount of micronized deutetrabenazine and a second pharmaceutically acceptable excipient coat; and optionally further comprising
e) a capsule shell or pharmaceutical sachet packaging.

56. The dosage form of Aspect 55, wherein the core comprises a) immediate release granules, immediate release pellet or immediate release tablet comprising first amount of micronized deutetrabenazine and a first pharmaceutically acceptable excipient or b) an inert particle coated with a first amount of micronized deutetrabenazine and the first pharmaceutically acceptable excipient.

57. The dosage form of Aspect 55 or Aspect 56, wherein the first and/or the second amount of pH-independent polymer coat, independently, comprises ethylcellulose, polyethylene glycol and triacetin, optionally further comprising povidone.

58. The dosage form of Aspect 55 or Aspect 56, wherein the first and/or the second amount of pH-independent polymer coat comprises cellulose acetate and optionally further comprising polyethylene glycol.

59. The dosage form of any one of the Aspects 1-53, comprising a population of sustained release beads and further comprising a population of immediate release beads, wherein the immediate release beads comprise a) immediate release granules, immediate release pellet or immediate release tablet comprising a first amount of micronized deutetrabenazine and a first pharmaceutically acceptable excipient or b) an inert particle coated with a first amount of micronized deutetrabenazine and a first pharmaceutically acceptable excipient.

60. The dosage form of Aspect 59, wherein the population of sustained release beads comprise:
a) a core comprising the first amount of micronized deutetrabenazine and the first pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate;
b) a first amount of pH-dependent polymer coat coating the core; and optionally further comprising
c) second amount of micronized deutetrabenazine and a second pharmaceutically acceptable excipient coat coating the first amount of the pH-dependent polymer coat; and optionally further comprising
d) a second amount of pH-dependent polymer coat coating the second amount of micronized deutetrabenazine and a second pharmaceutically acceptable excipient coat; and optionally further comprising
e) a capsule shell or pharmaceutical sachet packaging.

61. The dosage form of Aspect 60, wherein the core comprises a) immediate release granules, immediate release pellet or immediate release tablet comprising the first amount of micronized deutetrabenazine and the first pharmaceutically acceptable excipient or b) an inert particle coated with the first amount of micronized deutetrabenazine and the first pharmaceutically acceptable excipient.

62. The dosage form of Aspect 60 or Aspect 61, wherein the first and/or the second amount of pH-dependent polymer coat comprises methacrylic acid-ethyl acrylate copolymer, hydroxypropylmethyl cellulose phthalate (HPMCP), alginates, carboxymethylcellulose, or combinations thereof.

63. The dosage form of Aspect 62, wherein the first and/or the second amount of pH-dependent polymer coat comprises methacrylic acid-ethyl acrylate copolymer.

64. The dosage form of Aspect 61 or Aspect 62, wherein the first and/or the second amount of pH-dependent polymer coat comprises cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac, or combinations thereof.

65. The dosage form of Aspect 64, wherein the first and or the second amount of pH-dependent polymer coat comprises methacrylic acid-methyl acrylate copolymer.

66. The dosage form of Aspect 59, comprising the population of sustained release beads of Aspect 61 or Aspect 62.

67. The dosage form of Aspect 59, comprising the population of sustained release beads of Aspect 63 or Aspect 64.

68. The dosage form of Aspect 59, comprising the population of sustained release beads of Aspects 61 or Aspect 62; and further comprising the population of sustained release beads of Aspect 63 or Aspect 64.

69. The dosage form of any one of the previous Aspects in the form of a capsule or a pharmaceutical sachet package.

70. The dosage form of any one Aspects 1-69, wherein about 50% of micronized deutetrabenazine is released within 7 hours, as measured in a USPIII dissolution device, pH 7.2.

71. The dosage form of any of Aspects 1-70, for the use in the treatment of a VMAT2 mediated disorder.

72. A method of treating a VMAT2 mediated disorder comprising, orally administering to a patient in a need thereof, the controlled release dosage form of any of Aspects 1-70.

73. The dosage form of Aspect 71 or the method of Aspect 72, wherein the VMAT2 mediated disorder is hyperkinetic movement disorder.

74. The dosage form or the method of Aspect 73, wherein the hyperkinetic movement disorder is chronic hyperkinetic movement disorder.

75. The dosage form or the method of Aspect 74, wherein the chronic hyperkinetic movement disorder is selected from chorea associated with Huntington's disease, Tardive dyskinesia, and dyskinesia in cerebral palsy.

76. The dosage form or the method of any one of Aspects 71-75, wherein single dose administration of the oral dosage form comprising 6 mg of micronized deutetrabenazine provides an in vivo plasma profile for total α- and β-dihydrodeutetrabenazine that includes a geometric mean AUC0-inf of about 90,000 to 142,750 h*pg/mL and/or a geometric mean Cmax of less than about 4,600 pg/mL.

77. The dosage form or the method of any one of Aspects 71-75, wherein single dose administration of the oral dosage form comprising 12 mg of micronized deutetrabenazine provides an in vivo plasma profile for total α- and β-dihydrodeutetrabenazine that includes a geometric mean AUC0-inf of about 180,000 to 285,500 h*pg/mL and/or a geometric mean Cmax of less than about 9,200 pg/mL.

78. The dosage form or the method of any one of Aspects 71-75, wherein single dose administration of the oral dosage form comprising 24 mg of micronized deutetrabenazine provides an in vivo plasma profile for total α- and β-dihydrodeutetrabenazine that includes a geometric mean AUC0-inf of about 360,000 to 571,000 h*pg/mL and/or a geometric mean Cmax of less than about 18,400 pg/mL.

79. The dosage form or the method of any one of Aspects 71-75, wherein single dose administration of the oral dosage form comprising 36 mg of micronized deutetrabenazine provides an in vivo plasma profile for total α- and β-dihydrodeutetrabenazine that includes a geometric mean AUC0-inf of about 540,000 to 856,500 h*pg/mL and/or a geometric mean Cmax of less than about 27,600 pg/mL.

80. The dosage form or the method of any one of Aspects 71-75, wherein single dose administration of the oral dosage form comprising 48 mg of micronized deutetrabenazine provides an in vivo plasma profile for total α- and β-dihydrodeutetrabenazine that includes a geometric mean AUC0-inf of about 720,000 to 1,142,000 h*pg/mL and/or a geometric mean Cmax of less than about 36,800 pg/mL.

81. The dosage form or the method of any one of Aspects 71-75, wherein administration of the oral dosage form comprising 6 mg of micronized deutetrabenazine provides an in vivo plasma profile for total α- and β-dihydrodeutetrabenazine that includes a geometric mean AUC0-24 of about 102,500 to 200,000 h*pg/mL at steady state and/or a mean Cmax of less than about 10,000 pg/mL at steady state.

82. The dosage form or the method of any one of Aspects 71-75, wherein administration of the oral dosage form comprising 12 mg of micronized deutetrabenazine provides an in vivo plasma profile for total α- and β-dihydrodeutetrabenazine that includes a geometric mean AUC0-24 of about 205,000 to 400,000 h*pg/mL at steady state and/or a mean Cmax of less than about 20,000 pg/mL at steady state.

83. The dosage form or the method of any one of Aspects 71-75, wherein administration of the oral dosage form comprising 24 mg of micronized deutetrabenazine provides an in vivo plasma profile for total α- and β-dihydrodeutetrabenazine that includes a geometric mean AUC₀₋₂₄ of about 400,000 to 800,000 h*pg/mL at steady state and/or a mean Cₘₐₓ of less than about 40,000 pg/mL at steady state.

84. The dosage form or the method of any one of Aspects 71-75, wherein administration of the oral dosage form comprising 36 mg of micronized deutetrabenazine provides an in vivo plasma profile for total α- and β-dihydrodeutetrabenazine that includes a geometric mean AUC₀₋₂₄ of about 615,000 to 1,200,000 h*pg/mL at steady state and/or a mean Cₘₐₓ of less than about 60,000 pg/mL at steady state.

85. The dosage form or the method of any one of Aspects 71-75, wherein administration of the oral dosage form comprising 48 mg of micronized deutetrabenazine provides an in vivo plasma profile for total α- and β-dihydrodeutetrabenazine that includes a geometric mean AUC₀₋₂₄ of about 800,000 to 1,600,000 h*pg/mL at steady state and/or a mean Cₘₐₓ of less than about 80,000 pg/mL at steady state.

86. A process for manufacturing the core of the sustained release beads of any one of Aspects 1-70 or the immediate release beads of any one of Aspects 5-70, comprising the steps of;
a) providing a dispersion of a first amount of micronized deutetrabenazine with a first pharmaceutically acceptable excipient, wherein the pharmaceutically acceptable excipient comprises: an antioxidant, a binder, an anti-foaming agent, a filler, and a surfactant;
b) forming immediate release granules, immediate release pellet or immediate release tablet from the dispersion of a); or coating an inert particle with the dispersion of a);
thereby generating the immediate release beads or the core of the sustained release beads, respectively.

87. A process for manufacturing the sustained release beads of any one of Aspects 1-70, comprising the steps of:
a) providing a core, wherein the core comprises immediate release granules, immediate release pellet or immediate release tablet comprising a dispersion of a first amount of micronized deutetrabenazine and a first pharmaceutically acceptable excipient; or an inert particle coated with a dispersion of a first amount of micronized deutetrabenazine and a first pharmaceutically acceptable excipient;
b) coating the core of a) with a first coat selected from a pH-independent polymer coating, a pH-dependent polymer coating or with a pH-independent polymer coating and a pH-dependent polymer coating;
c) optionally further coating the first coat with a second amount of micronized deutetrabenazine and a second pharmaceutically acceptable excipient coat;
d) optionally further coating the beads of c) with a second coat selected from a pH-independent polymer coating, a pH-dependent polymer coating or with a pH-independent polymer coating and a pH-dependent polymer coating thereby generating sustained release beads.

88. The process of Aspect 87, wherein the process for preparing the core comprises the steps of
a) providing a dispersion of the first amount of micronized deutetrabenazine with a first pharmaceutically acceptable excipient, wherein the pharmaceutically acceptable excipient comprises: an antioxidant, a binder, an anti-foaming agent, a filler, and a surfactant;
b) forming immediate release granules, immediate release pellet or immediate release tablet from the dispersion of a); or coating an inert particle with the dispersion of a);
thereby generating the immediate release beads or the core of the sustained release beads, respectively.

89. The process of any one of Aspects 86-88, wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate 90. The process of any one of Aspects 86-89, further comprising coating the sustained release beads with a film coat, comprising a mixture of hydrophilic and hydrophobic polymers.

91. The process of Aspect 90, wherein the hydrophilic polymer comprises polyacrylic acid, polyvinyl alcohol, polyethylene glycol, polyvinyl pyrrolidone, polyethylene oxide, alginic acid and its salts, chitosan, carrageenan, gum Arabic, guar gum, agar agar, gelatin, xanthan, locust bean gum, methyl cellulose, carboxymethyl cellulose, hydroxyethyl cellulose, hydroxypropyl cellulose, hydroxypropyl methylcellulose, starches, and combinations thereof.

92. The process of Aspect 90, wherein the hydrophobic polymer comprises ethyl cellulose, cellulose acetate, cellulose acetate phthalate, cellulose acetate butyrate, shellac, methacrylate and acrylate copolymers (enteric and non-enteric), poly(lactic acid), poly(lactide-co-glycolide), hydroxypropyl methylcellulose phthalate, hydroxypropyl methylcellulose acetate succinate, poly(vinyl acetate), and combinations thereof.

1. A controlled release oral dosage form for twice daily administration of deutetrabenazine comprising a population of sustained release beads; wherein the sustained release beads comprise a core comprising
a first amount of micronized deutetrabenazine and a first pharmaceutically acceptable excipient, and
further comprising a first coat selected from a pH-independent polymer coat, a pH-dependent polymer coat, or a pH-independent polymer coat further coated with a pH-dependent polymer coat.

2. The dosage form of claim 1, wherein the core comprises
a) immediate release granules, immediate release pellet or immediate release tablet comprising the first amount of micronized deutetrabenazine and the first pharmaceutically acceptable excipient; or
b) an inert particle coated with the first amount of the micronized deutetrabenazine and the first pharmaceutically acceptable excipient.

3. The dosage form of claim 1, wherein the sustained release beads are further coated with a second amount of micronized deutetrabenazine and a second pharmaceutically acceptable excipient on top of the first coat.

4. The dosage form of claim 3, wherein the sustained release beads are further coated with a second coat selected from a pH-independent polymer coat, a pH-dependent polymer coat or a pH-independent polymer coat further coated with a pH-dependent polymer coat, on top of the second amount of the micronized deutetrabenazine and the second pharmaceutically acceptable excipient.

5. The dosage form of claim 1, further comprising a population of immediate release beads; wherein the population of immediate release beads comprises
a) immediate release granules, immediate release pellets or immediate release tablets comprising an immediate release amount of micronized deutetrabenazine and immediate release pharmaceutically acceptable excipient; or
b) an inert particle coated with an immediate release amount of micronized deutetrabenazine and an immediate release pharmaceutically acceptable excipient.

6. (canceled)

7. The dosage form of claim 1, wherein the pharmaceutically acceptable excipient in the first amount of micronized deutetrabenazine and the first pharmaceutically acceptable excipient and the second amount of micronized deutetrabenazine and the second pharmaceutically acceptable excipient and the immediate release amount of micronized deutetrabenazine and immediate release pharmaceutically acceptable excipient, are identical or
wherein the pharmaceutically acceptable excipient in the first amount of micronized deutetrabenazine and the first pharmaceutically acceptable excipient and the second amount of micronized deutetrabenazine and the second pharmaceutically acceptable excipient and the immediate release amount of micronized deutetrabenazine and immediate release pharmaceutically acceptable excipient, are different.

8. The dosage form of claim 1, wherein the micronized deutetrabenazine has a median particle size of 0.05 to 100 micron, or 1 to 30 micron, or 5 to 25 micron.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The dosage form of claim 1, wherein the sustained release beads comprise a first and optionally a second pH-independent polymer coat coating the core.

24. (canceled)

25. (canceled)

26. (canceled)

27. (canceled)

28. (canceled)

29. (canceled)

30. (canceled)

31. (canceled)

32. (canceled)

33. The dosage form of claim 1, wherein the dosage form comprises a total of 6 mg-72 mg of micronized deutetrabenazine.

34. The dosage form of any one of claim 1, consisting essentially of a population of sustained release beads.

35. The dosage form of claim 34 wherein the population of sustained release beads comprises:
a) a core comprising a first amount of micronized deutetrabenazine and a first pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate;
b) a first pH-independent polymer coat coating the core; and optionally further comprising
c) a second amount of micronized deutetrabenazine and a second pharmaceutically acceptable excipient coat coating the first pH-independent polymer coat; and optionally further comprising
d) a second pH-independent polymer coat coating the second amount of micronized deutetrabenazine and a second pharmaceutically acceptable excipient coat; and optionally further comprising
e) a capsule shell or pharmaceutical sachet packaging.

36. The dosage form of claim 35, wherein the core comprises a) immediate release granules, immediate release pellet or immediate release tablet comprising first amount of micronized deutetrabenazine and a first pharmaceutically acceptable excipient or b) an inert particle coated with a first amount of micronized deutetrabenazine and the first pharmaceutically acceptable excipient.

37. (canceled)

38. (canceled)

39. The dosage form of claim 1, comprising a population of sustained release beads and further comprising a population of immediate release beads, wherein the immediate release beads comprise a) immediate release granules, immediate release pellet or immediate release tablet comprising a first amount of micronized deutetrabenazine and a first pharmaceutically acceptable excipient or b) an inert particle coated with a first amount of micronized deutetrabenazine and a first pharmaceutically acceptable excipient.

40. The dosage form of claim 39, wherein the population of sustained release beads comprise:
a) a core comprising the first amount of micronized deutetrabenazine and the first pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate, mannitol, sodium bicarbonate and mixtures thereof, and a surfactant comprising sodium lauryl sulfate;
b) a first amount of pH-dependent polymer coat coating the core; and optionally further comprising
c) second amount of micronized deutetrabenazine and a second pharmaceutically acceptable excipient coat coating the first amount of the pH-dependent polymer coat; and
optionally further comprising
d) a second amount of pH-dependent polymer coat coating the second amount of micronized deutetrabenazine and a second pharmaceutically acceptable excipient coat; and
optionally further comprising
e) a capsule shell or pharmaceutical sachet packaging.

41. The dosage form of claim 40, wherein the core comprises a) immediate release granules, immediate release pellet or immediate release tablet comprising the first amount of micronized deutetrabenazine and the first pharmaceutically acceptable excipient or b) an inert particle coated with the first amount of micronized deutetrabenazine and the first pharmaceutically acceptable excipient.

42. (canceled)

43. (canceled)

44. (canceled)

45. (canceled)

46. (canceled)

47. (canceled)

48. (canceled)

49. (canceled)

50. The dosage form of claim 1, wherein about 50% of micronized deutetrabenazine is released within 4 hours, as measured in a USPIII dissolution device, pH 7.2.

51. (canceled)

52. A method of treating a VMAT2 mediated disorder comprising, orally administering to a patient in a need thereof, the controlled release dosage form of claim 1.

53. The method of claim 52, wherein the VMAT2 mediated disorder is hyperkinetic movement disorder.

54. The dosage form or the method of claim 53, wherein the hyperkinetic movement disorder is chronic hyperkinetic movement disorder.

55. The dosage form or the method of claim 54, wherein the chronic hyperkinetic movement disorder is selected from chorea associated with Huntington's disease, Tardive dyskinesia, and dyskinesia in cerebral palsy.
